(19) United States
(12) Patent Application Publication
(10) Pub. No.: US 2005/0192430 A1
(43) Pub. Date: Sep. 1, 2005

(54) Linker arms for nanocrystals and compounds thereof

(76) Inventors: Sandra J. Rosenthal, Nashville, TN (US); Ian D. Tomlinson, Nashville, TN (US); Tadd Kippeny, Corrales, NM (US)

Correspondence Address: STITES & HARBISON PLLC, 424 CHURCH STREET, SUITE 1800, NASHVILLE, TN, 37219-2376, US

(21) Appl. No.: 10/977,175

(22) Filed: Oct. 29, 2004

Related U.S. Application Data
(63) Continuation-in-part of application No. 09/864,728, filed on May 24, 2001, now abandoned.
(60) Provisional application No. 60/206,771, filed on May 24, 2000.

(51) Int. Cl. C07K 16/00

(52) U.S. Cl. 530/391.3

(57) ABSTRACT

A nanocrystal compound comprising: a nanocrystal, and attached thereto a compound of the following formula: 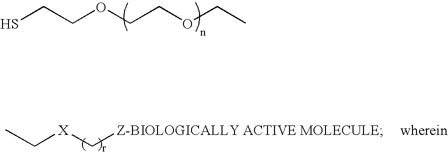
n is 0 or an integer from 1 to 48; X and Z are independently O, NH, N—R, S, CH₂, CO, COHN, NHCO, SO, SO₂NH, NHSO₂, carbamate and thio carbamate; R is alkyl or aryl; r is 0 or an integer from 1 to 15; and wherein S is the attachment point to a nanocrystal compound. The nanocrystal compounds of the present invention are useful fluorescent labels.

### PRIORITY

This application is a continuation-in-part of, and claims priority to, U.S. patent application Ser. No. 09/864,728, filed May 24, 2001, now abandoned, which claims priority to U.S. patent application Ser. No. 60/206,711, filed May 24, 2000, now abandoned. The contents of both applications are incorporated herein by reference.

### FIELD OF THE INVENTION

This invention generally relates to nanocrystals, linker arms for nanocrystals, and compounds resulting therefrom. Furthermore, this invention relates to labeling techniques using the compounds of the present invention.

### BACKGROUND OF THE INVENTION

Semi-conducting nanocrystals, also referred to as quantum dots, have many advantages over traditional dye molecules in the areas of fluorescent labeling. Fluorescent nanocrystal labeling has broad application in the biomedical sciences. For example, the labeling technique of the present invention provides improved and widely applicable methods for detecting biomolecules and for scrutinizing biomolecular processes.

Currently quantum dots are being used as fluorescent tags capable of tracing specific substances within cells. Quantum dots can be activated to glow with different colors, so it is easier to use quantum dots in tandem than combinations of conventional fluorescent dyes. See “Semiconductor Beacons Light up Cell Structures” Service, Science, Vol. 281. The conventional fluorescent dye, typically made from small organic dye molecules can be toxic, can quench quickly, and can be difficult to use in tandem, since typically each dye must be excited with photons at a different wavelength. Additionally, compared with conventional coloring agents such as rhodamine 6G or other organic dyes, the quantum dots produce narrower and much brighter fluorescence spectra. See “Quantum Dots Meet Biomolecules”, Jacoby. With the quantum dots, or nanocrystals, the absorbency onset and emission maxima shift to a higher energy with decreasing size. The excitation typically tracks the absorbency, resulting in a tunable fluorophore that can be excited efficiently at any wavelength shorter than the emission peak, yet will emit with the same characteristic a narrow, symmetric spectrum regardless of the excitation wavelength. See “Semiconductor Nanocrystals as Fluorescent Biological Labels”, Bruchez, et al., Science, Vol. 281, 1998. The absorbance onset and emission maximum shift to higher energy as the size of the nanocrystal decreases. Because the excitation tracks absorbance, the nanocrystals can be excited at many wavelengths, yet still they emit the same narrow, symmetric peak. By varying the material used or the size of the quantum dot, the color can be changed. Additionally, a range of quantum dots of different colors may be excited with a single wavelength and detected simultaneously. See “Bright Lights for Biomolecules”, Analytical Chemistry News and Features, December 1998. Thus, the quantum dots, or semiconducting nanocrystals, are much more flexible and advantageous when used in assays.

The attachment of biologically active ligands to nanocrystals including, for example, cadmium selenide nanocrystals, is a new method of producing novel fluorescent sensors. The sensors can have a variety of applications. They may be used in fundamental studies ranging from assay systems to locate the distribution and localization of membrane bound receptors, transporter proteins and channels in whole assay systems. They may also be used in novel methodologies for the development of pharmaceutically active compounds using high throughput screening.

The small size of the of the nanocrystal ligand conjugate offers advantages over conventional techniques that use antibodies bound to fluorescent dyes. These advantages include the small size of the drug nanocrystal conjugate which enables it to fit into the synaptic gap. Antibody-fluorescent dye systems are much larger than the nanocrystal drug conjugates of the present invention, so the antibody-fluorescent dye stems are less likely to fit into the synaptic gap. Additionally most antibodies are cell permeable.

The increased photostability of the nanocrystals means that they are not as easily photo-bleached as conventional dyes. Therefore, the nanocrystal compounds of the present invention may be used in experiments that require longer periods of illumination without photo-bleaching becoming a major problem.

The increased brightness of the nanocrystals enhances the sensitivity of the assay systems when compared to traditional dyes. Therefore, assay systems can be developed that detect lower concentrations of the analyte.

Also see “Quantum Dot Bioconjugates for Ultrasensitive Nonisotopic Detection”, Chan, Nie, Science, Vol. 281, 1998.

There are several patents that disclose nanocrystals that can be used in connection with the present invention.

U.S. Pat. No. 5,990,479 to Weiss et al. discloses a luminescent nanocrystal compound that is capable of linking to an affinity molecule. Weiss et al. further describe a process for making luminescent semiconductor nanocrystal compounds and for making an organo luminescent semiconductor probe comprising the nanocrystal compound linked to an affinity molecule capable of bonding to a detectable substance and a process for using the probe to determine the presence of a detectable substance in a material.

U.S. Pat. No. 5,751,018 to Alivisatos et al. discloses methods for attaching semiconductor nanocrystals to solid inorganic surfaces, using self-assembled bifunctional organic monolayers as bridge compounds.

U.S. Pat. No. 5,537,000 to Alivisatos et al., which describes electroluminescent devices formed using semiconductor nanocrystals as an electron transport media and a method for making such electroluminescent devices.

U.S. Pat. No. 5,505,928 to Alivisatos et al. discloses nanocrystals of III-V semiconductors, and U.S. Pat. No. 5,262,352 Alivisatos et al. discloses a process for forming a solid, continuos thin film of a semiconductor material on a solid support surface.

Additionally, the highly fluorescent cadmium selanide/zinc sulfide core shell nanocrystals have many desirable qualities as imaging agents in biological assay systems. Their large quantum yields, photostability and narrow emission spectra will enable the development of fluorescent assay systems to image live cell cultures. As their fluorescent emission spectra is size tunable it will be possible to image several different biological targets simultaneously in order to understand their interactions in live cell cultures. The absorption spectra of nanocrystals is a continuum above the first band gap thus only a single light source is required to excite several colors. This property will enable the development of low cost high through put assay systems that don't require radio labeled materials.

### SUMMARY OF THE INVENTION

An aspect of the present invention is to provide linker arms to attach organic compounds to nanocrystals, or quantum dots. Another aspect of the present invention is quantum dot compounds. Another aspect of the present invention is methods of using the quantum dot compounds of the present invention.

Generally speaking, the compounds of the present invention are of the following formula:

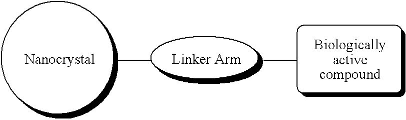

Examples of the biologically active compounds of the present invention include seratonin or seratonin derivatives, cocaine analogues, phenyl tropane analogues, phenylisopropylamine derivatives, dopamine derivatives, melatonin derivatives, chlormethiazole derivatives, derivatives of RTI-4229-75, and derivatives of GBR 12935. RTI-4229-75 and GBR 12935 are further described below.

For the purposes of providing examples only, the following are examples of organic compounds attached to nanocrystal as described in the present invention:

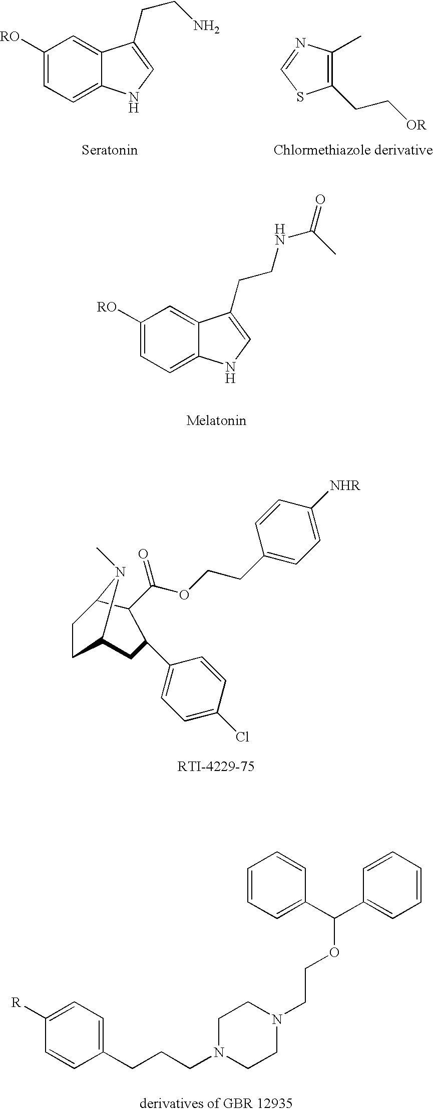

In the above examples, R represents the attachment point to the linker arm. Additionally, the R group may be “floating” when attached to the phenyl ring. That is, the R group may be attached to any available carbon atom on the ring.

The present invention further is directed to nanocrystal compounds, which include linker arm derivatives of the present invention. More specifically, the nanocrystal compounds of the present invention comprise a semiconducting nanocrystal and a linking arm having a first portion linked to the nanocrystal and a second portion linked to an organic compound.

### BRIEF DESCRIPTION OF THE DRAWINGS

FIG. 1 shows a biologically active water soluble core/shell nanocrystal of the present invention.

FIG. 2 shows an amphiphilic polymer/TOPO coated core shell nanocrystal.

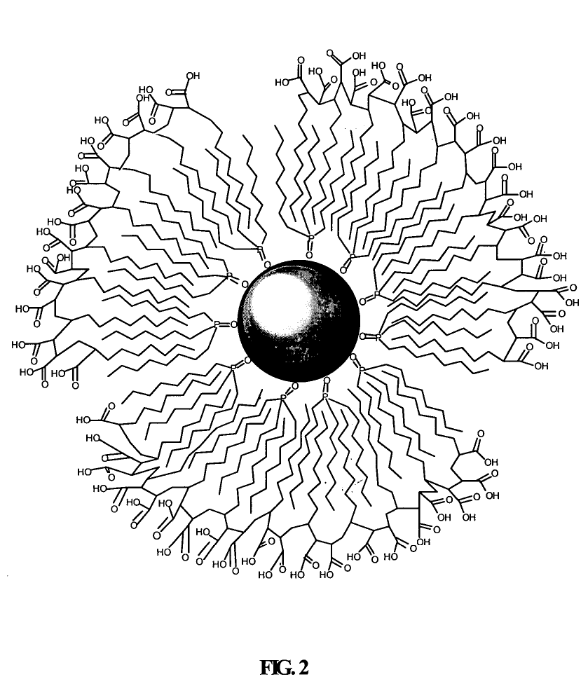

FIG. 3 shows a pegilated amphiphilic polymer/TOPO coat ed core shell nanocrystal. N is 10 or greater, X a reactive group such as OH, NH₂, COOH, etc.

### DETAILED DESCRIPTION OF THE INVENTION

As stated above, the present invention relates to linker arms to which biologically active molecules can be attached to nanocrystals. The nanocrystals used in conjunction with the present invention are the nanocrystals typically used in fluorescent imaging techniques. Preferably, the nanocrystals used in conjunction with the present invention are semiconductor nanocrystals capable of luminescence and/or scattering or diffraction when excited by an electromagnetic radiation source (of broad or narrow bandwidth) or a particle beam, and capable of exhibiting a detectable change of absorption and/or emitting radiation in a narrow wavelength band and/or scattering or diffracting when excited. For exemplary purposes, the nanocrystals of U.S. Pat. No. 5,990,479 may be used with the present invention.

That is, in embodiments of the present invention, an organic or inorganic single crystal particle having an average cross-section of about 20 nanometers (nm) or 20×10⁻⁹ meters (200 Angstroms), preferably no larger than about 10 nm (100 Angstroms) and a minimum average cross-section of about 1 nm, although in some instances a smaller average cross-section nanocrystal, i.e., down to about 0.5 nm (5 Angstroms), may be acceptable. Typically the nanocrystal will have an average cross-section ranging in size from about 1 nm (10 Angstroms) to about 10 nm (100 Angstroms).

Furthermore, for exemplary purposes only, these nanocrystals include, but not are limited to CdSe, CdS, PbSe, PbS, and CdTe.

As mentioned above, there are disadvantages to traditional dye molecules that are used in the area of fluorescent labeling. For example, simultaneous localization of several different proteins in situ is currently limited by the wide emission spectra and photostabilities of fluorescent dyes traditionally used to study cell surface receptors, ion channels, and transporters. The nanocrystal compounds of the present invention can overcome the above deficiencies. For example, in one embodiment of the present invention, the nanocrystal compounds comprise core (CdSe)/shell(ZnS) semiconducting nanocrystals. Through quantum confinement, the fluorescent wavelength of these nanocrystals are continuously tunable by size. For example a 25 Angstrom nanocrystal of this embodiment emits at 455 nm while a 60 Angstrom nanocrystal of this embodiment emits at 625 nm. Unlike dye molecules and variants of green fluorescent protein, these nanocrystals have narrow gaussian emission spectra enabling multiplex imaging. The absorption of these nanocrystals is continuous above the band-gap; hence all sizes of nanocrystals can be excited with a single excitation wavelength. In addition, the nanocrystals of this embodiment are much brighter than traditional dyes, even hours after continuous illumination.

The present invention further relates to multiple organic compounds in combination with the linker arms of the present invention. The present invention further relates to a method of attaching a linker arm to multiple organic compounds and a method of attaching a linker arm to a nanocrystal. The present invention further relates to the linker arms herein described and nanocrystals attached to the linker arms herein described. The present invention also relates to nanocrystals and semiconductor nanocrystals in combination with the linker arms of the present invention. The present invention further relates to the attachment of a nanocrystal and a linker arm to an organic compound. The present invention relates to assay systems and assay kits for CNS research, receptor purification, pathogens, environmental contaminants, toxins, and screening for drugs, insecticides, herbicides, and other biologically active substances.

The linker arms and linker arm compound derivatives of the present invention enhance stability and are relatively stable, including stability to biological degradation. The linker arms and the linker arm compound derivatives of the present invention are also advantageous in that they can be synthesized at a relatively low cost.

More specifically, the present invention relates to linker arms such as, for example, ether-containing, polyether or carbon-carbon chain linker arms by which biologically active molecules such as CNS drugs and neurotransmitters can be attached to nanocrystals. The attachment of a linker arm of the present invention allows nanocrystals to be used as imaging agents in diverse applications such as biochemical research, CNS research, receptor purification, and high throughput screening for new drugs and other biologically active substances.

Additionally, the present invention relates to linker arms such as, for example, ether containing, polyether or carbon linker arm by which biologically active molecules such as drugs, hormones, etc. can be attached to nanocrystals. The linker arms of the present invention enhance water solubility of nanocrystals and allow nanocrystals to be attached to a diverse range of molecules ranging from drugs to polypeptides and neurotransmitters. The linker arm compounds of the present invention allow nanocrystals to be used as imaging agents in diverse applications such as CNS research, receptor purification, assay systems for pathogens, environmental contaminants, toxins, and a high throughput assay system for new drugs and biologically active molecules.

As stated above, preferably the organic part of the nanocrystal compounds of the present invention are biologically active compounds. Preferably, the biologically active compound is one that will bind to detectable substances, if the substance is present, in the material being analyzed.

In general, any affinity molecule useful in the prior art in combination with a dye molecule to provide specific recognition of a detectable substance will find utility in the formation of the organo-luminescent semi conductor nanocrystal probes of the invention. Such affinity molecules include, by way of example only, such classes of substances as monoclonal and polyclonal antibodies, nucleic acids (both monomeric and oligomeric), proteins, polysaccharides, and small molecules such as sugars, peptides, drugs, and ligands. Lists of such affinity molecules are available in the published literature such as, by way of example, the “Handbook of Fluorescent Probes and Research Chemicals”, (sixth edition) by R. P Haugland, available from Molecular Probes, Inc.

As stated above, the compounds of the present invention enable nanocrystals to be used as probes for neurotransmitters, receptors and transporter proteins. In one embodiment of the present invention, seratonin (5-hydroxytriptamine) is attached to a nanocrystal. Seratonin is a neurotransmitter which has been linked to the regulation of critical behaviors including sleep, appetite, and mood.

The seratonin transporter (SERT) is a 12-transmembrane domain protein responsible for the uptake of seratonin by the cell. The seratonin labeled nanocrystal compounds of the present invention have a measurable ability to block the uptake of tritiated sepatonin by the human and Drosophila seratonin transporter (hSERT and dSERT).

Seratonin labeled nanocrystals (SNACs) of the present invention may be prepared by reacting trioctylphosphineoxide coated nanocrystals with seratonin and tetramethylammonium hydroxide in methanol. The SNACs are isolated by precipitation and purified to remove seratonin. Linkage of the seratonin presumptively occurs through the lone pair of the hydroxyl to the Cd surface atoms of the nanocrystal. hSERT and dSERT are transfected into HeLa cells via a vaccinia virus/T7 expression system. Following expression of the transfected transporters, the cells are assayed for uptake of tritiated seratonin in the presence of increasing concentrations of SNACs. Kᵢ values, the concentration at which half the SNACs are bound to the transporter, are determined by nonlinear regression. The values [Kᵢ(hSERT)=74 uM, Kᵢ(dSERT)=29 uM] indicate SNACs can effectively interact with the seratonin recognition site of the transporter.

These results suggest that highly fluorescent, seratonin labeled nanocrystals can be used as probes for SERT. These probes assist in determining the structure of SERT, including the number of gene products (SERT proteins) that are required to assemble a functional unit, and following transporter movement within the cell.

The present invention enables nanocrystals to be used as imaging agents, which results in an assay system that is superior to traditional immunoassay systems because, among other things, several wavelengths can be used to induce fluorescence. The linker arm can be attached to a number of different ligands, thus enabling them to be used in high throughput screening and receptor purification. The linker arm is stable and not as subject to enzymatic degradation as other linker arms may experience. The linker arm of the present invention also enhances the solubility of the nanocrystal, and can be readily derivitised. This enables a wide range of molecules to be attached to the nanocrystals. The linker arm of the present invention is not as temperature sensitive as many immunoassay systems, and thus is likely to have a longer shelf life. Further, the linker arm of the present invention is also robust and therefore not susceptible to extremes of pH that may denature and degrade peptide linkers.

To use core/shell nanocrystals as a biological imaging agent cadmium selanide/zinc sulfide core shell nanocrystals have to be derivitised to make them water soluble and a biologically active ligand has to be attached to confer biological activity. Cadmium selanide/zinc sulfide core shell nanocrystals are frequently synthesized with trioctyl phosphine oxide (TOPO) bound to their surfaces. TOPO is not necessary for biological activity. To make the nanocrystals water soluble most of the TOPO must be displaced by a water soluble ligand. Several methods for making nanocrystals water soluble exist. One method frequently used is to replace the TOPO on the surface with mecapto acetic acid. When synthesizing mercapto acetic acid coated nanocrystals the TOPO may be displaced by pyridine. This is subsequently displaced by mercapto acetic acid. Ligands terminated with thiols such as compounds (I) through to (XIV) described in this patent may be conjugated directly to the surface of the nanocrystal in conjunction with mercapto acetic acid. Resulting in biologically active nanocrystals with solubility in water. The linker arms of these ligands can be modified to increase the water solubility and stability of the colloidal suspension. Also the length of the linker arm may be changed to increase the biological affinity of the nano conjugates for their target receptors. Conjugates such as these may be used to image transfected cells expressing the appropriate receptor or transporter protein or image neuronal cell cultures as well as in novel high through put assay systems.

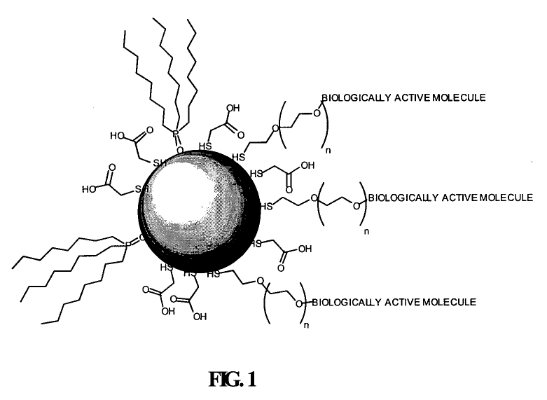

The biologically active molecule shown in FIG. 1 may be a drug or neurotransmitter. The PEG chain may either be attached directly to the biologically active molecule via a covalent bond or it may be attached to a short alkyl spacer the other end of which is attached to the biologically active molecule of interest. The length of this spacer may be between 2 and 15 carbon atoms and the length of the PEG chain attached to the spacer or biologically active molecule may be between 2 and 50 ethylene glycol units long. The thiol at the end of the PEG chain is attached to the surface of the nanocrystal.

Embodiments of the biologically active ligands of the present invention are represented by the following formula and analogs and isomers thereof: 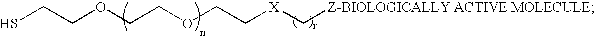
wherein n is a number between 0 and 48. X represents a point of attachment to the poly ethylene glycol chain. The polyethylene glycol chain is either linked via X to a alkyl chain or to a biologically active molecule via Z which may be one of the following functionalities, O, NH, NR, S, CH₂, CO, COHN, NHCO, SO, SO₂NH, NHSO₂, carbamate and thio carbamate. R may be either an alkyl substituent or an aryl substituent. X may be O, NH, NR, S, CH₂,CO, COHN, NHCO, SO, SO₂NH, NHSO₂, carbamate and thio carbamate. R may be either an alkyl substituent or an aryl substituent. r may be either 0 or have a value between 2 and 15. S is the attachment point to a nanocrystal compound.

Specific ligands include the below compoounds. Additionally, nanocrystal compounds of the present invention comprise a nanocrystal and the following compounds, with S being the attachment point to the nanocrystal: 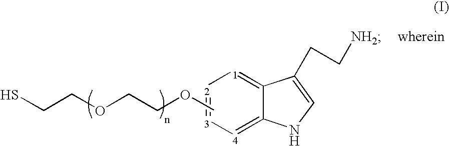
n is 0-10, and the linker arm may be attached to positions 1, 2, 3, or 4. In other aspects, the linker arm is attached to position 2. Also, in other embodiments, n is 2, 3, 4, or 5. 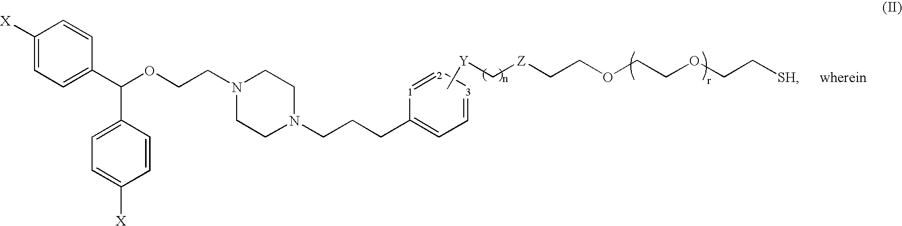
r is 1-10; Z is O, S, NH, CH₂, CONH, NHCO, NH, SO, SO₂NH, NHSO₂, carbamate, thiocarbamate, NH—R(R is aryl or alkyl); n is 1-15; Y is O, S, NH, CH₂, CONH, NHCO, NH, SO, SO₂NH, NHSO₂, carbamate, thiocarbamate, NH—R(R is aryl or alkyl). X is H or halo. The linker arm may be attached to positions 1, 2 or 3. In other embodiments, the linker arm is attached to position 3. Also, X may be halo, including F. n may 4, 5, 6, 7, 8, 9, or 10. r may be 2, 3, 4, or 5. 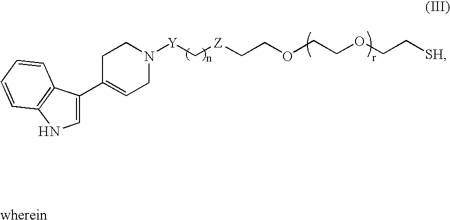
r is 1-10 Z is O, S, NH, CH₂, CONH, NHCO, NH, SO, SO₂NH, NHSO₂, carbamate, thiocarbamate, NH—R(R is aryl or alkyl). n is 1-15. In certain embodiments, n is 4,5,6,7,8,9, or 10. In certain embodiments, r is 2, 3, 4, or 5. Y is O, S, NH, CH₂, CONH, NHCO, NH, SO, SO₂NH, NHSO₂, carbamate, thiocarbamate, NH—R(R is aryl or alkyl). 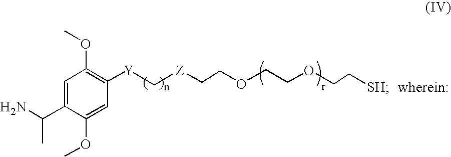
r is 1-10. In certain embodiments, r is 2, 3, 4, or 5. Z is O, S, NH, CH₂, CONH, NHCO, NH, SO, SO₂NH, NHSO₂, carbamate, thiocarbamate, NH—R(R is aryl or alkyl). n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NH—R(R is aryl or alkyl). 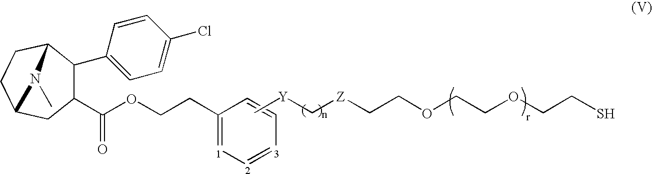
r is 1-10. In certain embodiments, r is 2, 3, 4, or 5. Z is O, S, NH, CH₂, CONH, NHCO, NH, SO, SO₂NH, NHSO₂, carbamate, thiocarbamate, NH—R(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NH—R(R is aryl or alkyl). The linker arm may be attached to positions 1, 2 or 3. Preferably position 3. 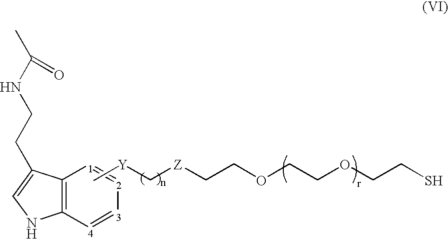
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 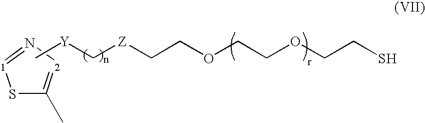
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1 or 2. Preferably position 2. 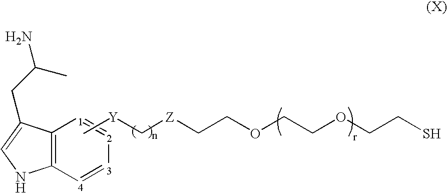
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 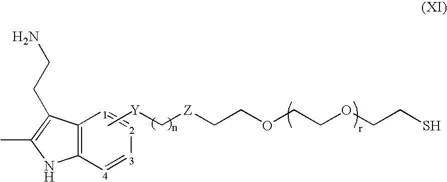
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 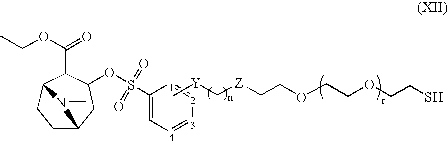
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1, 2 or 3. Preferably position 3. 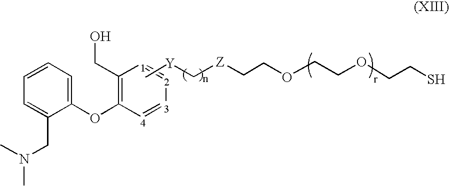
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 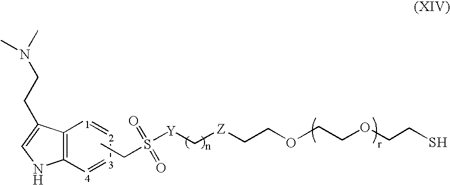
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2.

As used herein, the term alkyl or alkyl group is to be understood in the broadest sense to mean hydrocarbon residues which can be linear, i.e., straight-chain, or branched, and can be acyclic or cyclic residues or comprise any combination of acyclic and cyclic subunits. Further, the term alkyl as used herein expressly includes saturated groups as well as unsaturated groups which latter groups contain one or more, for example, one, two, or three, double bonds and/or triple bonds.

All these statements also apply if an alkyl group carries substituents or occurs as a substituent on another residue, for example, in an alkyloxy residue, or an arylalkylamino residue. Examples of alkyl residues containing from 1 to 20 carbon atoms are methyl, ethyl, propyl, butyl, pentyl, hexyl, heptyl, octyl, nonyl, decyl, undecyl, dodecyl, tetradecyl, hexadecyl, octadecyl, and eicosyl, the n-isomers of all these residues, isopropyl, isobutyl, 1-methylbutyl, isopentyl, neopentyl, 2,2-dimethylbutyl, 2-methylpentyl, 3-methylpentyl, isohexyl, 2,3,4-trimethylhexyl, isodecyl, sec-butyl, tert-butyl, or tert-pentyl.

Unsaturated alkyl residues are, for example, alkenyl residues such as vinyl, 1-propenyl, 2-propenyl (═allyl), 2-butenyl, 3-butenyl, 2-methyl-2-butenyl, 3-methyl-2-butenyl, 5-hexenyl, or 1,3-pentadienyl, or alkynyl residues such as ethynyl, 1-propynyl, 2-propynyl (═propargyl), or 2-butynyl. Alkyl residues can also be unsaturated when they are substituted.

Examples of cyclic alkyl residues are cycloalkyl residues containing 3, 4, 5, 6, 7, or 8 ring carbon atoms like cyclopropyl, cyclobutyl, cyclopentyl, cyclohexyl, cycloheptyl, or cyclooctyl which can also be substituted and/or unsaturated. Unsaturated cyclic alkyl groups and unsaturated cycloalkyl groups like, for example, cyclopentenyl or cyclohexenyl can be bonded via any carbon atom. The term alkyl as used herein also comprises cycloalkyl-substituted alkyl groups like cyclopropylmethyl-, cyclobutylmethyl-, cyclopentylmethyl-, cyclohexylmethyl-, cycloheptylmethyl-, cyclooctylmethyl-, 1-cyclopropylethyl-, 1-cyclobutylethyl-, 1-cyclopentylethyl-, 1-cyclohexylethyl-, 1-cycloheptylethyl-, 1-cyclooctylethyl-, 2-cyclopropylethyl-, 2-cyclobutylethyl-, 2-cyclopentylethyl-, 2-cyclohexylethyl-, 2-cycloheptylethyl-, 2-cyclooctylethyl-, 3-cyclopropylpropyl-, 3-cyclobutylpropyl-, 3-cyclopentylpropyl-, 3-cyclohexylpropyl-, 3-cycloheptylpropyl-, or 3-cyclooctylpropyl- in which groups the cycloalkyl subgroup as well as acyclic subgroup also can be unsaturated and/or substituted.

Of course, a group like (C₁-C₈)-alkyl is to be understood as comprising, among others, saturated acyclic (C₁-C₈)-alkyl, (C₃-Cg)-cycloalkyl, cycloalkyl-alkyl groups like (C₃-C₇)-cycloalkyl-(C₁-C₅)-alkyl- wherein the total number of carbon atoms can range from 4 to 8, and unsaturated (C₂-C₈)-alkyl like (C₂-C₈)-alkenyl or (C₂-C₈)-alkynyl. Similarly, a group like (C₁-C₄)-alkyl is to be understood as comprising, among others, saturated acyclic (C₁-C₄)-alkyl, (C₃-C₄)-cycloalkyl, cyclopropyl-methyl-, and unsaturated (C₂-C₄)-alkyl like (C₂-C₄)-alkenyl or (C₂-C₄)-alkynyl.

Unless stated otherwise, the term alkyl preferably comprises acyclic saturated hydrocarbon residues containing from 1 to 6 carbon atoms which can be linear or branched, acyclic unsaturated hydrocarbon residues containing from 2 to 6 carbon atoms which can be linear or branched like (C₂-C₆)-alkenyl and (C₂-C₆)-alkynyl, and cyclic alkyl groups containing from 3 to 8 ring carbon atoms, in particular from 3 to 6 ring carbon atoms. A particular group of saturated acyclic alkyl residues is formed by (C₁-C₄)-alkyl residues like methyl, ethyl, n-propyl, isopropyl, n-butyl, isobutyl, sec-butyl, and tert-butyl.

The alkyl groups of the present invention can in general be unsubstituted or substituted by one or more, for example, one, two, three, or four, identical or different substituents. Any kind of substituents present in substituted alkyl residues can be present in any desired position provided that the substitution does not lead to an unstable molecule. Examples of substituted alkyl residues are alkyl residues in which one or more, for example, 1, 2, 3, 4, or 5, hydrogen atoms are replaced with halogen atoms.

Examples of substituted cycloalkyl groups are cycloalkyl groups which carry as substituent one or more, for example, one, two, three, or four, identical or different acyclic alkyl groups, for example, acyclic (C₁-C₄)-alkyl groups like methyl groups. Examples of substituted cycloalkyl groups are 4-methylcyclohexyl, 4-tert-butylcyclohexyl, or 2,3-dimethylcyclopentyl.

The term aryl refers to a monocyclic or polycyclic hydrocarbon residue in which at least one carbocyclic ring is present. In a (C₆-C₁₄)-aryl residue from 6 to 14 ring carbon atoms are present. Examples of (C₆-C₁₄-aryl residues are phenyl, naphthyl, biphenylyl, fluorenyl, or anthracenyl. Examples of (C₆-C₁₀)-aryl residues are phenyl or naphthyl. Unless stated otherwise, and irrespective of any specific substituents bonded to aryl groups, aryl residues including, for example, phenyl, naphthyl, and fluorenyl, can in general be unsubstituted or substituted by one or more, for example, one, two, three, or four, identical or different substituents. Aryl residues can be bonded via any desired position, and in substituted aryl residues the substituents can be located in any desired position.

In monosubstituted phenyl residues, the substituent can be located in the 2-position, the 3-position, or the 4-position, the 3-position and the 4-position being preferred. If a phenyl group carries two substituents, they can be located in 2,3-position, 2,4-position, 2,5-position, 2,6-position, 3,4-position, or 3,5-position. In phenyl residues carrying three substituents, the substituents can be located in 2,3,4-position, 2,3,5-position, 2,3,6-position, 2,4,5-position, 2,4,6-position, or 3,4,5-position. Naphthyl residues can be 1-naphthyl and 2-naphthyl. In substituted naphthyl residues, the substituents can be located in any positions, for example, in monosubstituted 1-naphthyl residues in the 2-, 3-, 4-, 5-, 6-, 7-, or 8-position and in monosubstituted 2-naphthyl residues in the 1-, 3-, 4-, 5-, 6-, 7-, or 8-position. Biphenylyl residues can be 2-biphenylyl, 3-biphenylyl, or 4-biphenylyl. Fluorenyl residues can be 1-, 2-, 3-, 4-, or 9-fluorenyl. In monosubstituted fluorenyl residues, bonded via the 9-position the substituent is preferably present in the 1-, 2-, 3-, or 4-position.

Unless stated otherwise, substituents that can be present in substituted aryl groups are, for example, (C₁-C₈)-alkyl, in particular (C₁-C₄)-alkyl, such as methyl, ethyl, or tert-butyl, hydroxy, (C₁-C₈)-alkyloxy, in particular (C₁-C₄)-alkyloxy, such as methoxy, ethoxy, or tert-butoxy, methylenedioxy, ethylenedioxy, F, Cl, Br, I, cyano, nitro, trifluoromethyl, trifluoromethoxy, hydroxymethyl, formyl, acetyl, amino, mono- or di-(C₁-C₄)-alkylamino, ((C₁-C₄)-alkyl)carbonylamino like acetylamino, hydroxycarbonyl, ((C₁-C₄)-alkyloxy) carbonyl, carbamoyl, optionally substituted phenyl, benzyl optionally substituted in the phenyl group, optionally substituted phenoxy, or benzyloxy optionally substituted in the phenyl group.

The above statements relating to aryl groups correspondingly apply to divalent residues derived from aryl groups, i.e., to arylene groups like phenylene which can be unsubstituted or substituted 1,2-phenylene, 1,3-phenylene, or 1,4-phenylene, or naphthalene which can be unsubstituted or substituted 1,2-naphthalenediyl, 1,3-naphthalenediyl, 1,4-naphthalenediyl, 1,5-naphthalenediyl, 1,6-naphthalenediyl, 1,7-naphthalenediyl, 1,8-naphthalenediyl, 2,3-naphthalenediyl, 2,6-naphthalenediyl, or 2,7-naphthalenediyl.

The above statements also correspondingly apply to the aryl subgroup in arylalkyl- groups. Examples of arylalkyl- groups which can also be unsubstituted or substituted in the aryl subgroup as well as in the alkyl subgroup, are benzyl, 1-phenylethyl, 2-phenylethyl, 3-phenylpropyl, 4-phenylbutyl, 1-methyl-3-phenyl-propyl, 1-naphthylmethyl, 2-naphthylmethyl, 1-(1-naphthyl)ethyl, 1-(2-naphthyl)ethyl, 2-(1-naphthyl)ethyl, 2-(2-naphthyl)ethyl, or 9-fluorenylmethyl.

Ligands of the present invention include the following compounds. Additionally, like above, nanocrystal compounds of the present invention include the following examples, with S being the attachment point of the nanocrystal:

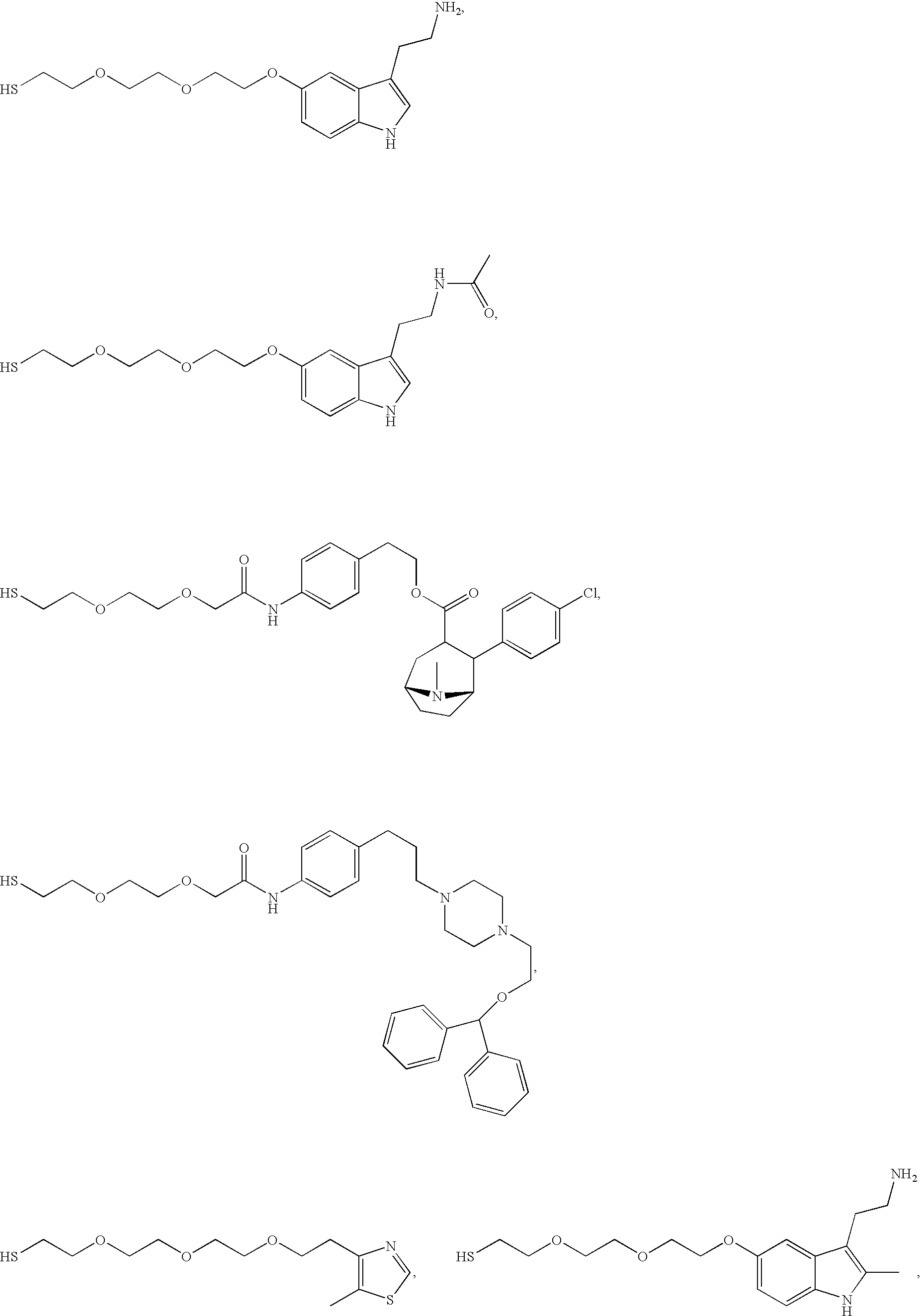

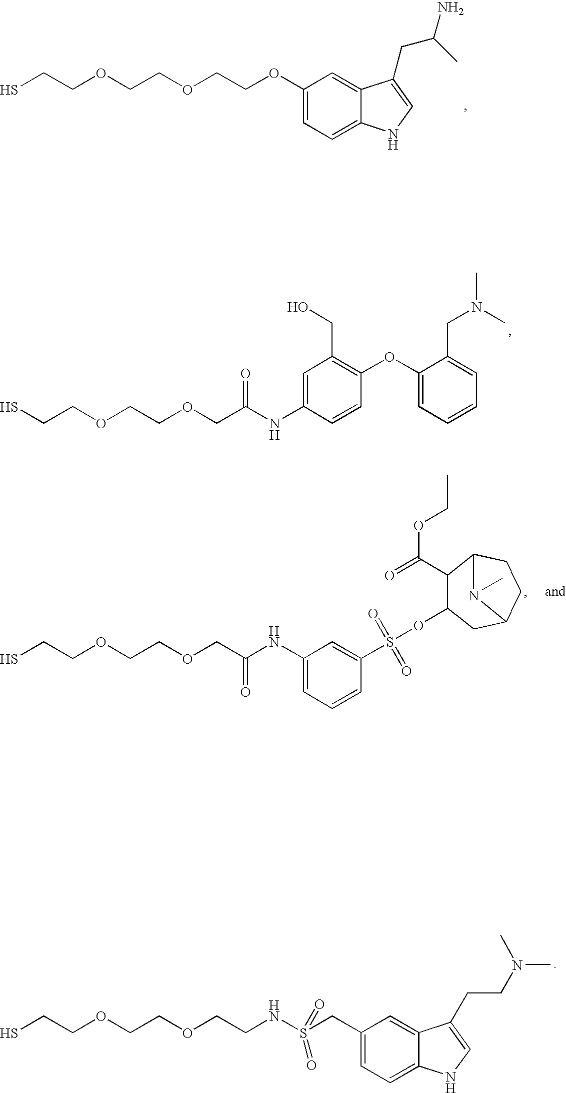

With respect to the compounds of the present invention, it is understood that the nanocrystals may have more than one ligand attached thereto, as is made clear in the figures. Thus, the nanocrystals described herein are interpreted as comprising the corresponding ligand as a substituent, not as consisting of a nanocrystal, the corresponding ligand, and a biologically active molecule.

Nanocrystal compounds of the present invention include compounds that comprise of nanocrystals with the following specific and preferred features: a CdSe core, ZnS shell, generally their cores are less than 25 nm, in diameter. The surrounding ZnS shell is typically 10 to 20 nm in thickness, and the ligand coated core shells are water solubilized by the addition of a mercapto acetic acid co-solubility ligand.

By attaching antibodies to nanocrystals via a linker arm of the present invention, nanocrystals can be made to bind to specific antigens. Accordingly, an embodiment of the present invention is an assay kit developed for the detection of a diverse range of substances ranging from environmental contaminant such as DDT, dioxanes, chemical warfare agents, herbicides, pesticides, and pathogenic organisms such as Ecoli 0157 and Salmonela.

For example, the present invention comprises a process for treating a material, such as a biological material, to determine the presence of a detectable substance in the material. The process comprises contacting the material with a nanocrystal conjugated compound of the present invention, washing unbound nanocrystal conjugated compound away, and exposing the material to energy such as an electromagnetic source or particle beam capable of exciting the nanocrystal conjugated compound of the present invention, and causing a detectable fluorescence to occur in the nanocrystal conjugated compound of the present invention. Thus enabling the location and distribution of a particular substance within the biological material to be determined.

The nanocrystal compounds of the present invention may be used in the assays described in U.S. Pat. No. 5,990,479.

One assay system of the present invention is a high throughput fluorescence assay to identify novel ligands that might be effective antidepressants or ligands that might help combat cocaine addiction. In this assay a known agonist or antagonist for the dopamine receptor or transporter is bound to nanocrystals, and incubated with cells that either naturally express or have been engineered to express dopamine receptors or transporters. After incubating for 12 hours excess ligands are removed by washing and unknown compounds are incubated with the cells for a further 12 hours. The cells are washed again with buffer and a fluorescence assay is performed. Any cells that no longer fluorescence have a high affinity ligand bound to them and this ligand may be used as a lead compound for drug discovery. Such an assay system may be carried out in a conventional multiple well format system, such as the 96 well format.

Chart A, below demonstrates another method of the present invention that may be used to detect biologically active analytes. Chart A describes a sandwich assay system. In chart A, in step 1 monoclonal or polyclonal antibodies raised against a specific analyte or groups of analytes are bound to the surface of the plate. In step 2, the analyte is added and binds to the antibody. In step 3, the unbound analyte is washed away and a nanocrystal antibody conjugated using our linker arm of the present invention is added (once again poly or monoclonal antibodies may be used). In step 3, the unbound nanocrystal antibody conjugates are removed by washing, and a fluorescence assay is performed to determine if the analyte is present in the sample being analyzed and its concentration as a sample with a higher concentration will produce a greater fluorescence. Multiple analytes can be screened for using a conventional 96 well plate format.

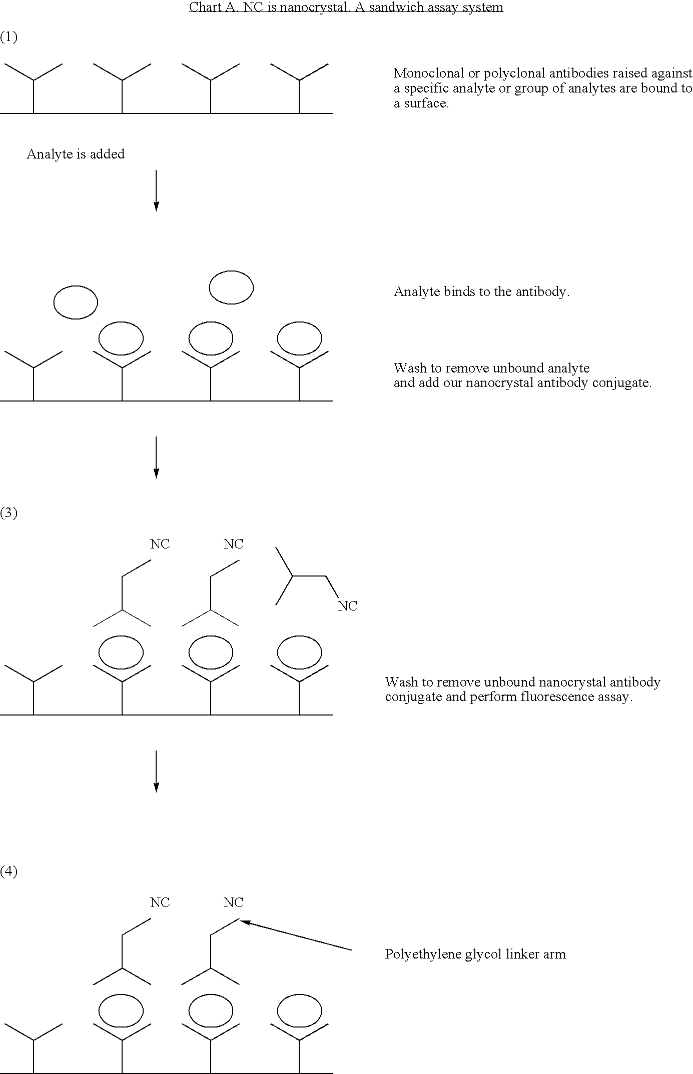

The nanocrystal of the present invention may be used in affinity chromatography, where a compound or biological molecule of interest may be bound to a column. This may then be specifically labeled with the antibody nanocrystal conjugate, substrate nanocrystal conjugate, or drug nanocrystal conjugate of the present invention. The compound could be a drug, a hormone, an enzyme, a protein, a nucleic acid or a receptor. Once the nanocrystal conjugate has bound to the substrate of interest, it may either remain bound to the column or be eluted with the mobile phase. This would enable the isolation and identification of the compound orbiological molecule of interest. Unlike fluorescent dyes, nanocrystals are not easily photo-bleached. Therefore, it would be easier to watch the compound or compounds eluting off the column. Also such a system may be applied to several different analytes enabling the identification of several unknowns at once by using different sized nanocrystals conjugated to different ligands. Thus it is theoretically possible to identify different receptor classes or subtypes (e.g. 5-HT receptor subtypes) as they elute off the column. For example it may be possible to differentiate between 5HT2 and 5HT3 receptor subtypes using such a system.

The linker arm acts as a spacer and separates the ligand from the nanocrystal thus possible steric and other interactions between nanocrystals and ligand are minimized. The linker arm may be an ethylene glycol moiety this helps to enhance the solubility in aqueous media. Many affinity chromatographic systems are typically run in such media. The polyether linker arm is also resistant to proteolytic cleavage which may be a problem with other assay systems.

Nanocrystals can be attached to enzymes via linker arms of the present invention. Thus the amino derived carboxylic acid derived poly-ethers may be linked to the backbone of the peptide via a peptide bond.

In this instance the linker arm removes the enzyme from the immediate environment of the nanocrystal. This may be important in reducing any effects that the nanocrystal may have upon the enzymes activity. Many such instances could be envisaged particularly if the enzyme or protein undergoes a conformational change during its catalytic cycle (e.g. Hemoglobin). Also the linker arm may increase the catalytic efficiency of the enzyme if the active site or sites are close to the enzymes surface.

Such a system may also be used to identify analytes in a similar manner to the nanocrystal antibody conjugates previously described. It may also be used in high throughput screening where the compounds of interest are bound to wells in plates and the enzyme nanocrystal conjugate is added. An example of this is shown in chart B below:

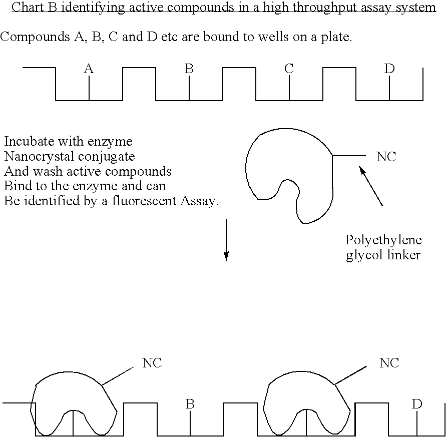

The enzymes substrate or inhibitor may also be bound to the polyethylene glycol nanocrystal conjugate. In this instance, the linker arm of the present invention reduces steric hindrance between nanocrystal and enzyme and it enables the substrate to enter the enzymes catalytic or alosteric site, which may not be possible if the substrate were bound to the surface of the nanocrystal (particularly if the site of interest is deep within the enzyme). An assay system that could use this technique as a tool for identifying new drugs is outlined in chart C, below, where compounds that will compete for the site of interest can be identified. If the nanocrystal is bound to an inhibitor via the linker arm of the present invention it is likely that this assay system could also be used to identify other inhibitors of the enzyme.

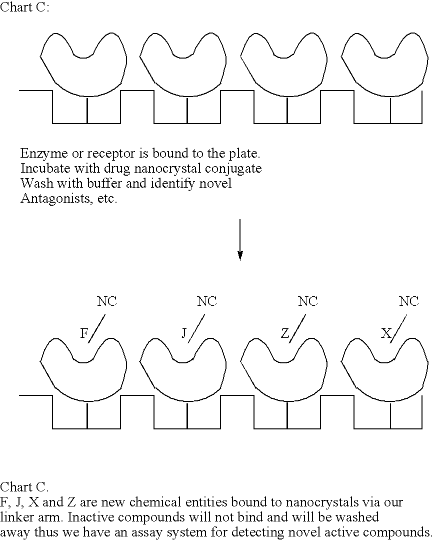

One specific substance may also be bound to the nanocrystal (e.g. a substrate for the enzyme) and a simple competitive assay could be performed with unknown substances in a manner similar to that shown above in chart C. Any substance that has a higher affinity for the site of interest on the enzyme, protein or receptor than the ligand conjugated nanocrystal would displace the ligand conjugated nanocrystal resulting in a loss off fluorescence, thus enabling this system also to be used as a high throughput assay system as well as an analytical tool for environmental contaminates, toxins, and other unknowns.

This system can be applied to receptors rather than enzymes. In this case, the nanocrystal is bound to an agonist, antagonist, or natural ligand for the receptor (e.g. Seratonin). This system could be used as an assay system for receptor agonist or antagonist. It would be of interest in neuropharmacology where receptor location and distribution could be mapped. By attaching different sized nanocrystals to different agonists, antagonists, or ligands it may be feasible to develop multiplexing assay systems, thus enabling the effects of drugs and other neurologically active agents to be monitored in whole cell assay systems. Assaying the location and distribution of many membrane bound receptors and transporter proteins is currently difficult using conventional antibody fluorescent dye systems is difficult due to photo-bleaching and the broader emission spectra of dyes.

Nanocrystals may be attached to DNA or RNA via the linker arm of the present invention. In this case, the major role of the linker arm acts as a spacer and reduces steric hindrance. The DNA or RNA conjugates may be used as a tool in molecular biology for identifying the location and frequency and rate of expression of specific gene sequences. Such a system is outlined in chart D, below:

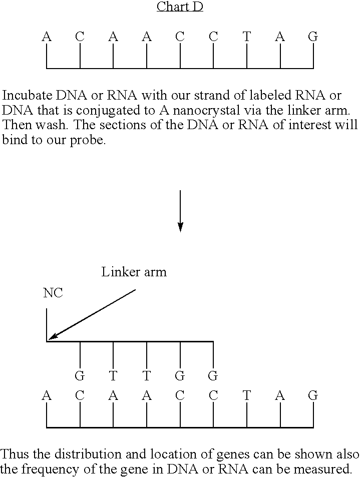

The nanocrystal conjugates of the present invention can also be used in assay systems in the same manner that antibody fluorescent dye conjugates, radio immuno assays, and ELISA are used. Examples of the assay system include routine assays used in medical laboratories such as tests for various disease states for example HIV, Diabetes, etc.

Another aspect of the present invention is biologically active nanocrystal conjugates based upon amphiphilic conjugated TOPO coated nanocrystals. These conjugates differ from the mercapto acetic acid water soluble conjugates. In order to make these nanocrystals water soluble the TOPO is not displaced by a water soluble ligand. Instead the TOPO is encapsulated in an amphiphlic polymer. Amphiphilic polymer coated core/shell nanocrystals have several advantages when compared to the mercapto acetic acid water soluble nanocrystals. These advantages include greater stability in solution at higher lower pH's, increased stability in solutions with high ionic strengths and higher quantum yields. The properties of core/shell nanocrystals coated with amphiphilic polymer should enable the development of biologically active nanocrystals which display even greater sensitivity. Thus enabling the imaging of cells with lower levels of expression of receptors and proteins. The fluorescent intensity of these nanocrystal is significantly greater than conventional dyes.

Amphiphilic polymer coated nanocrystals utilize a different methodology to produce water soluble nanocrystals. In these systems the TOPO isn't displaced it is left bound to the surface of the nanocrystal and an aphiphilic polymer consisting of hydrophobic chains bound to a hydrophilic polymer. Hydrophobic interactions between the hydrophobic chains and the TOPO result in a brush-like arrangement between the TOPO and the chains with the hydrophilic part of the polymer coating the surface of the dot.

Frequently the hydrophilic part of the polymer consists of poly carboxylic moieties. This gives high water solubility to the nanocrystal and the negative charge on the dot reduces aggregation and precipitation as each nanocrystal repels other nanocrystals in the solution.

Nanocrystals modified with amphiphilic coatings have greater stabilities than mercapto acetic acid coated nanocrystals. Their increased quantum yields when compared with mercapto acetic acid coated core shell nano crystals is due to the thiols being bound directly to the surfaces of dots. Thiols are known to act as traps, thiols bound to the surface of the nanocrystal can trap either the electron or hole released in the excitation of the nanocrystal. This reduces the brightness of the fluorescence of the mercapto acetic acid nanocrystals relative to TOPO coated nanocrystals.

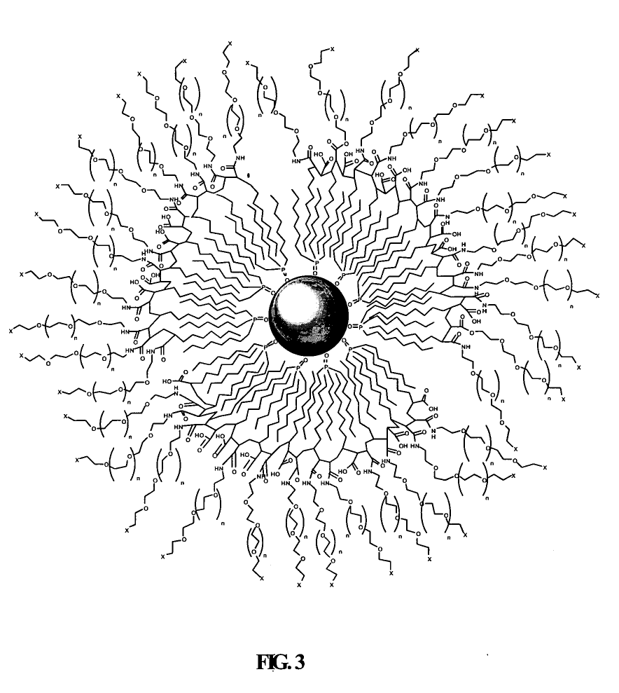

Ligands may be attached directly to the carboxyl groups of the amphiphilic polymer on the core however frequently the polymer is modified by adding a polyethylene glycol spacer. A diagram of a pegilated amphiphilic core shell nanocrystal is shown in FIG. 3. Typically the poly ethylene glycol is at least 12 monomers long. Thus n has a value of 10 or more. The substituent X is a reactive functionality that may be reacted with a ligand. X may include OH, NH₂, COOH, SH, etc. Pegilation increases the stability of the amphiphilic polymer coated dot by increasing the water solubility and it reduces non specific binding to cell surfaces.

The following is an example of specific routes for biologically active molecule/linker arm attachment.

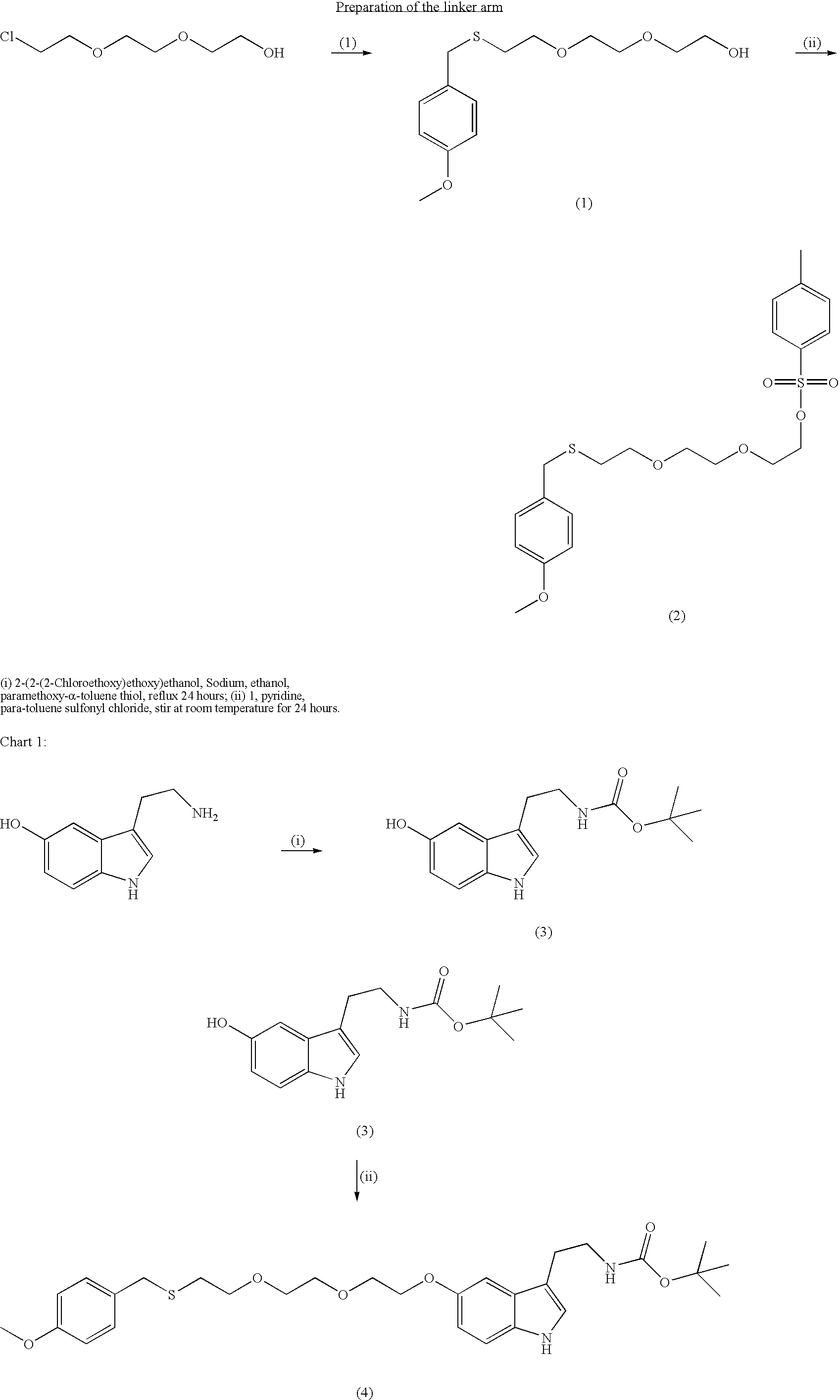

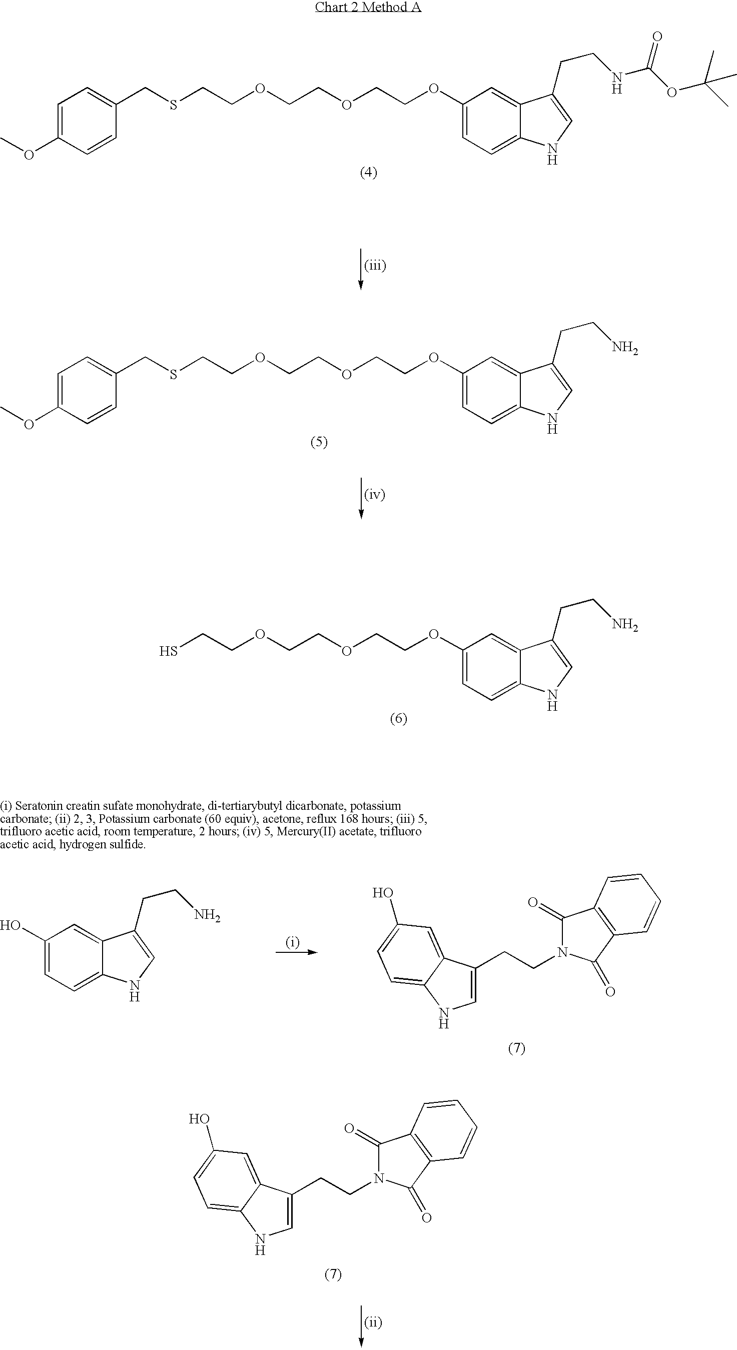

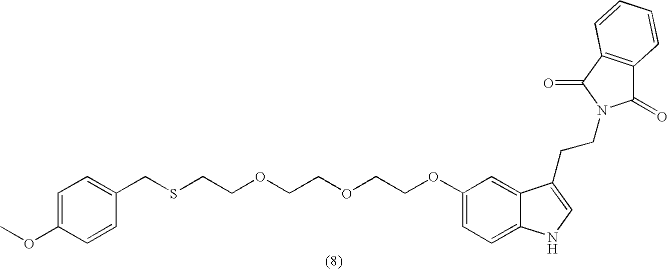

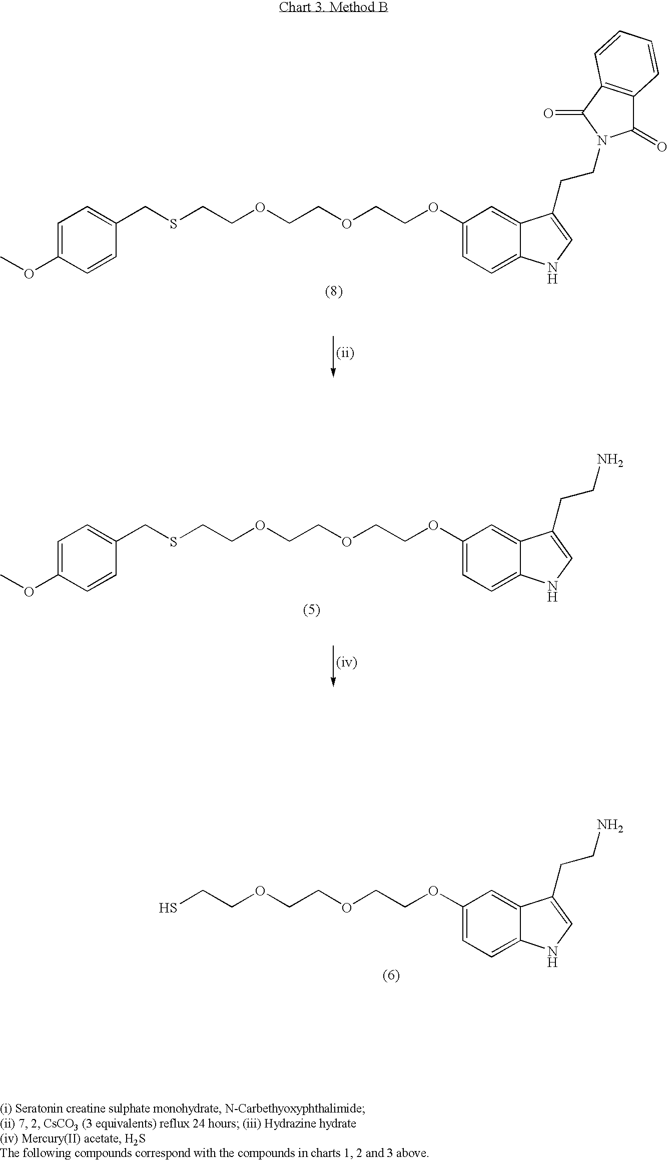

### 8-(4-\dot{m}ethoxybenzylthio)-3,6-dioxaoctanol (1)

Sodium metal (0.8 g, 34.8 mmols) is added to ethanol (100 ml) in a 250 ml round bottomed flask equipped with a reflux condenser and a stirrer. 4-Methoxy-α-toluenethiol (4.88 ml, 34.8 mmol) is added upon complete reaction of the sodium with the ethanol. The mixture is stirred at room temperature for 30 minutes then 2-(2-(2-chloroethoxy)ethoxy)ethanol (5.88 g, 34.8 mmols) is added. The reaction mixture is heated at reflux for 24 hours. After cooling to room temperature it is poured into saturated ammonium chloride solution (40 ml) and extracted into dichloromethane (3×100 ml). The dichloromethane solution is dried over magnesium sulfate and yields a yellow oil upon evaporation. The product is purified using column chromatography on silica eluted with a gradient system from dichloromethane to dichloromethane: methanol 10% to give approximately 7.24 g (71%) of the product as a yellow oil.

### 8-(4-Methoxybenzylthio)-3,6-dioxaoctyl tosylate (2)

8-(4-methoxybenzylthio)-3,6-dioxaoctanol (7.24 g, 25 mmols) is added to dry pyridine (5 ml) and cooled to 0° C. under nitrogen in a 100 ml flask equipped with a stirrer. Para-toluene sulphonyl chloride (6.5 g, 34 mmol) is slowly added to this solution and the mixture is stirred and allowed to warm to room temperature. It is stirred at room temperature for 18 hours after which it is added to water (100 ml) and dichloromethane (100 ml). The organic layer is separated and washed with hydrochloric acid (2N, 1×50 ml) and saturated sodium bicarbonate solution (50 ml). It is dried over magnesium sulfate, filtered and evaporated. The crude product is obtained as a red oil and this is purified using column chromatography, in which the crude material is adsorbed onto silica and the column is eluted with a gradient system running from 20% diethyl ether: petroleum ether to 70% diethyl ether: petroleum ether. This yields approximately 8.00 g (72%) of the product as a yellow oil.

### 3-[2-N-(tert-Butoxycarbonyl)amino]1H-indole-5-ol (3)

This compound is prepared as previous described in the Journal of medicinal chemistry 1996, 39, 314, Glennon R., et al. Potassium carbonate (1.3 g, 9.5 mmols) is added all at once to a suspension of seratonin creatine sulfate monohydrate (1.9 g, 4.7 mmols) dissolved in water (24 ml), in a 100 ml flask equipped with a stirrer. When the materials have dissolved di-tertbutyl dicarbonate (1.01 g, 4.7 mmols) is added. The mixture is left stirring at room temperature for 24 hours. The product is extracted with ethyl acetate (3×20 ml). The combined organic extracts are washed with water (1×20 ml), hydrochloric acid (5%, 15 ml) and brine (15 ml). The organic solution is dried over magnesium sulfate and the crude product is obtained as a black tar upon evaporation. The product is purified using column chromatography on silica gel eluted with dichloromethane. This yields approximately 1.34 g (100%) of the product as a pale yellow oil.

### 1-[3-[2-[N-(tert-Butoxycarbonyl)amino]ethyl]-1H-indol-5-yloxy]-3,6-dioxa-8-(4-methoxybenzylthio)octane (4)

8-(4-Methoxybenzylthio)-3,6-dioxaoctyl tosylate (2.82 g, 6.4 mmols) is added to acetone (100 ml). 3-[2-N-(tert-Butoxycarbonyl)amino] 1H-indole-5-ol (1.76 g, 6.4 mmols) is dissolved in acetone (20 ml) and the two solutions are combined in a 250 ml flask equipped with a reflux condenser and a stirrer. Dry potassium carbonated (60 g) is added and the mixture is left refluxing for 168 hours. Upon cooling the solution is filtered and evaporated. The product is purified using column chromatography on silica gel eluted with a gradient system running from dichloromethane to 90% dichloromethane: methanol. This gave 1.9 g (60%) of the product as a yellow oil.

### 1-[3-[2-aminoethyl]-1H-indol-5-yloxyl]-3,6-dioxa-8-(4-methoxybenzylthio) octane (5)

Method A:

1-[3-[2-[N-(tert-Butoxycarbonyl)amino]ethyl]-1H-indol-5-yloxy]-3,6-dioxa-8-(4-methoxybenzylthio)octane (0.8 g, 1.5 mmols) is dissolved in toluene (60 ml) in a 250 ml round bottomed flask equipped with a stirrer. Trifluoro acetic acid (20 ml) is added and the mixture is stirred at room temperature for 2 hours. It is evaporated under reduced pressure and the product is purified using silica gel chromatography eluted with triethylamine 3%: methanol 5%: dichloromethane 92%. This yields approximately 0.6 g (92%) of the product (5) as a pale yellow oil.

Method B:

1-[3-[2-[N,N-Phtalimido]ethyl]-1H-indol-5-yloxyl]-3,6-dioxa-8-(4-methoxybenzylthio) octane (1.4 g, 2.4 mmols) is dissolved in absolute ethanol (50 ml) in a 100 ml round bottomed flask equipped with a stirrer. Hydrazine mono hydrate (2 ml) is added and the solution is stirred for 18 hours at room temperature and then evaporated. Dichloromethane (50 ml) is added to the resulting tar and the mixture is heated at reflux for 30 minutes. After cooling it is filtered and evaporated and 7 was purified using silica gel column chromatography eluted with dichloromethane 95%: triethylamine 3%: methanol 2%. This yields approximately 0.6 g (51%) of the product (5) as a pale yellow oil.

### 1-[3-[2-amino ethyl]-1H-indol-5-yloxy]-3,6-dioxa-8-mercapto octane (6)

1-[3-[2-aminoethyl]-1H-indol-5-yloxyl]-3,6-dioxa-8-(4-methoxybenzylthio) octane (0.6 g, 1.4 mmols) is dissolved in trifluoroacetic acid (15 ml) and cooled to 0° C. in a 50 ml round bottomed flask equipped with a stirrer. Anisole (1.5 ml) and mercury (II) acetate (0.516 g, 1.6 mmols) are added and the mixture is stirred at 0° C. for 2 hours. The solution is evaporated and the resulting solid is washed with diethyl ether (3×50 ml). After air drying the solid is dissolved in glacial acetic acid (25 ml) and hydrogen sulfide is bubbled through the solution for 30 minutes. Mercuric sulfide is removed by filtration and the solution is evaporated to dryness. The resulting oil is dissolved in dichloromethane and washed with sodium bicarbonate solution (1M, 1×20 ml). The solution is dried over magnesium sulfate and evaporated. This yields approximately 0.072 g (39%) as a pale yellow oil.

### N,N-Phthalimido-2-(5-hydroxy-1H-indole-3-yl)ethylamine (7)

This compound is prepared using the method that has previously been described in the Journal of medicinal chemistry 1996, 39, 4717 Barf T., et. al. To a stirred solution of seratonin creatine sulphate monohydrate (3.8 g, 9.4 mmols) in water (15 ml) and tetrahydro furan (15 ml), is added a solution of 10% sodium bicarbonate until a pH of 8 is obtained. N-Carbethoxyphthalimide is added and the mixture is stirred at room temperature overnight. The resulting solid is removed by filtration and it is re-crystallized from absolute ethanol, to give 2.5 g (87%) of product as a yellow solid. Mp 215-216° C. (lit 213-216° C.).

### 1-[3-[2-[N,N-Phtalimido]ethyl]-1H-indol-5-yloxyl]-3,6-dioxa-8-(4-methoxybenzylthio)octane (8)

8-(4-Methoxybenzylthio)-3,6-dioxaoctyl tosylate (1.6 g, 3.6 mmols) is added to acetone (100 ml) then N,N-Phthalimido-2-(5-hydroxy-1H-indole-3-yl)ethylamine (1 g, 3.3 mmols) is added. Dry Cesium carbonate (3 g, 3 equivalents) was added and the mixture is heated at reflux for 24 hours. The solution is cooled to room temperature and filtered. The product (8) is purified using silica gel eluted with dichloromethane 98%: methanol. This gives approximately 1.1 g of (8) as a pale yellow oil.

### Attaching the Linker Arm to Alkyl Alcohols

The linker arms of the present invention may be attached to alkyl alcohols via an ether linkage. Many drugs, DNA, RNA, glycoproteins, intracellular messengers and hormones such as the steroids contain these functionalities. By way of an example a derivative of the neuroprotective agent chlormethiazole (9) has been synthesized. 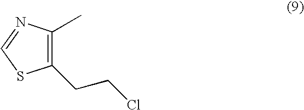
A synthesis of the derivative of chlormethiazole is outlined in chart 4, below. 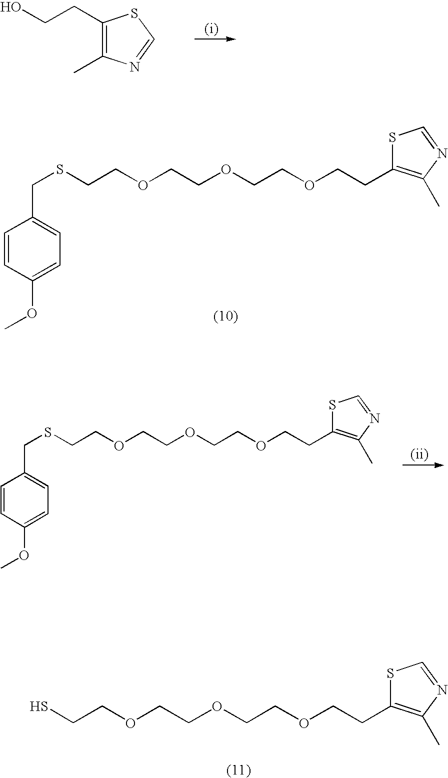
Chart 4. (i) 4-methyl-5-thiazoleethanol, 2, KOH, tertiary butyl ammonium chloride; (ii) Mercury(II) acetate, trifluoroacetic acid, Hydrogen sulfide.

### 2-[2-[2-[2-(4-Methyl-thiazol-5-yl)-ethoxy]ethoxy]ethoxy]thioethyl-(4-methoxybenzyl)ether (10)

4-methyl-5-thiazoleethanol (1.5 ml, 13.2 mmols) is added to dichloromethane (50 ml). Potassium hydroxide (4 g) dissolved in water (4 ml) and tertiary butyl ammonium chloride (0.02 g) are added. 8-(4-Methoxybezylthio)-3,6-dioxaoctyl tosylate (1.8 g, 4.4 mmols) is dissolved on dichloromethane and added to the mixture. The mixture is heated at reflux for 240 hours and then cooled to room temperature. Water (20 ml) is added, the organic layer is separated and dried over magnesium sulfate.

After removing the magnesium sulfate by filtration the solvent is removed under reduced pressure. The product is purified using column chromatography on silica gel eluted with ethyl acetate 99%: methanol. This yields approximately 0.33 g (20%) of the product as a pale yellow oil.

### 2-[2-[2-[2-(4-Methyl-thiazol-5-yl)ethoxy]ethoxy]ethoxy]ethanethiol (11)

2-[2-[2-[2-(4-Methyl-thiazol-5-yl)-ethoxy]ethoxy]ethoxy]thioethyl-(4-methoxybenzyl)ether (0.33 g, 0.9 mmols) is dissolved in trifluoroacetic acid (10 ml) and cooled to 0° C. When the solution is at a temperature of 0° C. mercury (II) acetate (0.3 g, 0.9 mmols) and anisole (1 ml) are added and the mixture is stirred at 0° C. for 2 hours. The solvent is evaporated under reduced pressure and the mercury salt is triturated with diethyl ether (3×50 ml). The resulting solid is dissolved in glacial acetic acid (20 ml) and hydrogen sulfide is bubbled through the solution for 30 minutes. After which the solution is filtered and evaporated under reduced pressure. The product is purified via column chromatography on silica gel eluted with dichloromethane 99%: methanol. This yields approximately 0.02 g (8%) of the product as a colorless oil.

### Alteration of Linker Arm to Attach Aryl and Alkyl Amines via an Amide Linkage

The polyethylene glycol linker arm can be altered so that it can be attached to aryl and alkyl amines via an amide linkage. The derivative of the linker arm can be readily prepared and a synthetic scheme for the derivative is outlined in chart 5, below.

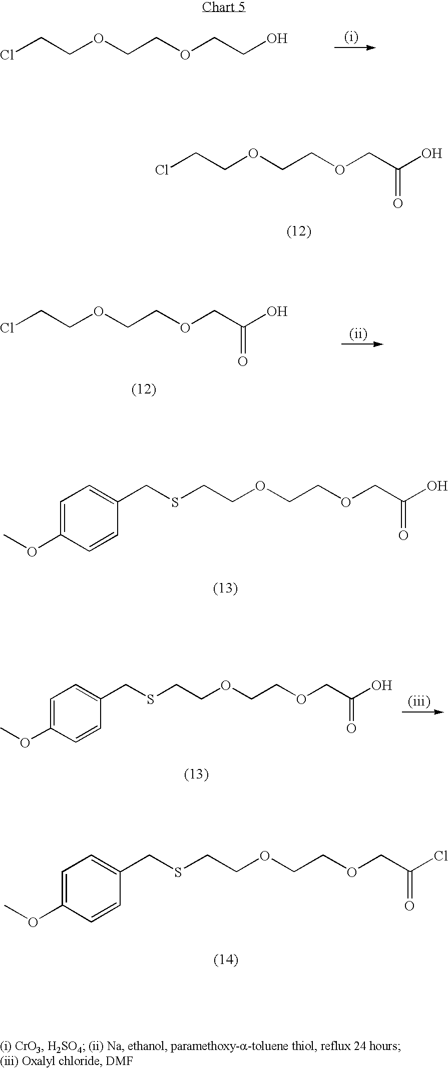

The resulting carboxylic acid (13) can be attached to amines using a variety of reagents such as DCC or by making the acid chloride (14). Two such derivatives that we have synthesized are the derivative of the cocaine analogue RTI-4229-75 (15) and the derivative of GBR 12935 (16):

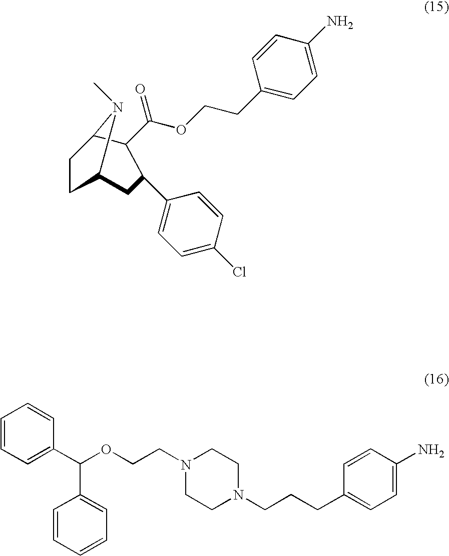

The synthesis of these compound are outlined in charts 6, 7 and 8, below. The linker arm derivative that contains this carboxylic acid functionality may also be attached to proteins and antibodies via an amide bond, alternatively it may be attached to RNA and DNA via a ester linkage to the ribose or deoxy ribose moiety.

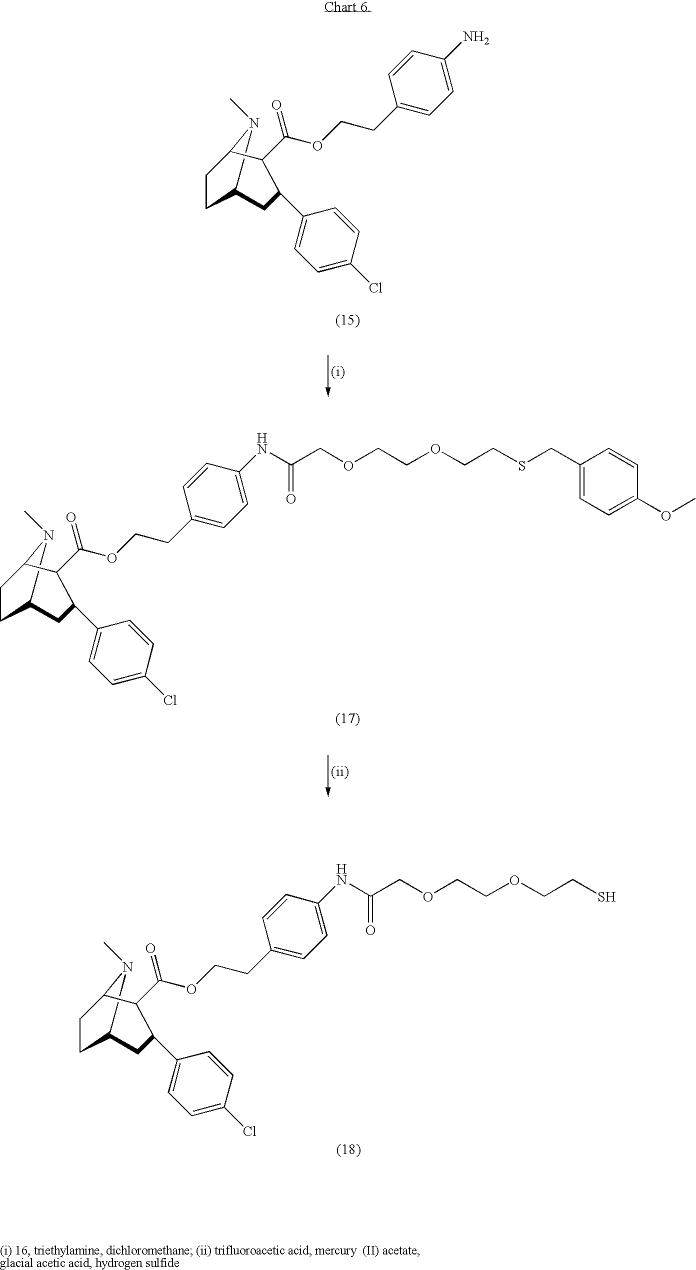

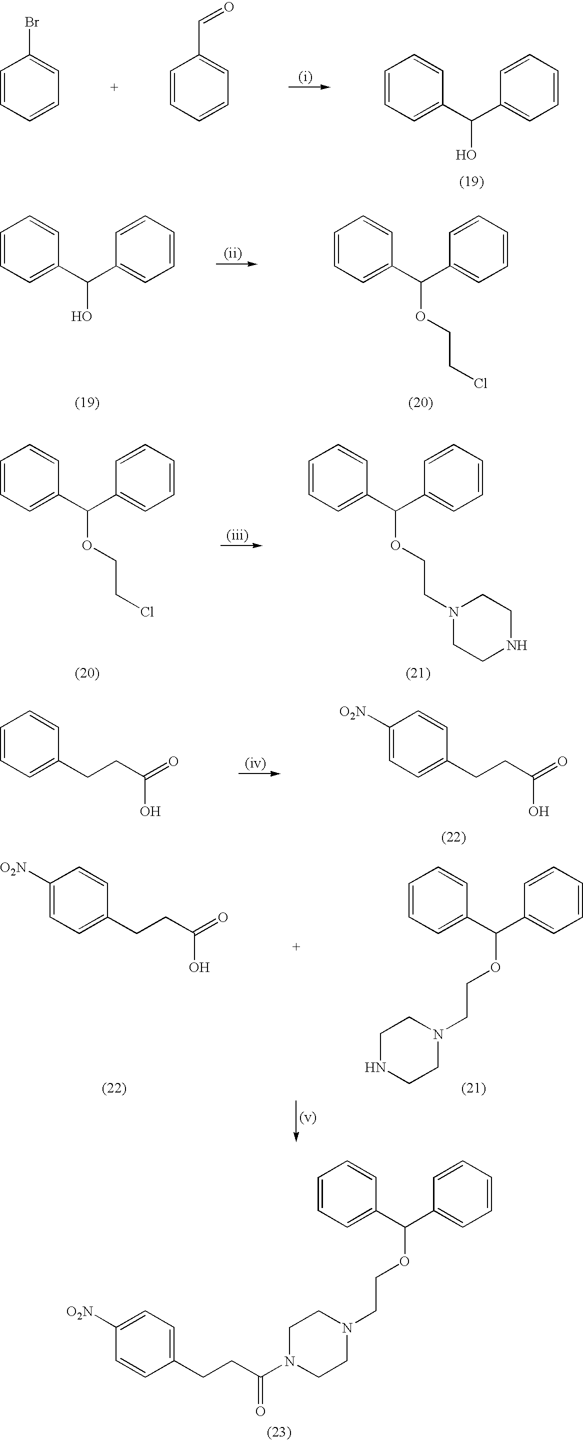

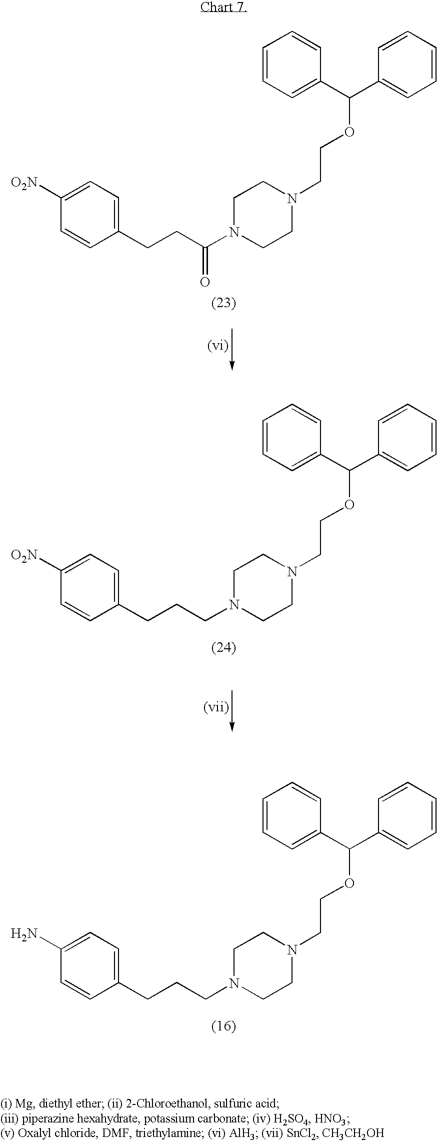

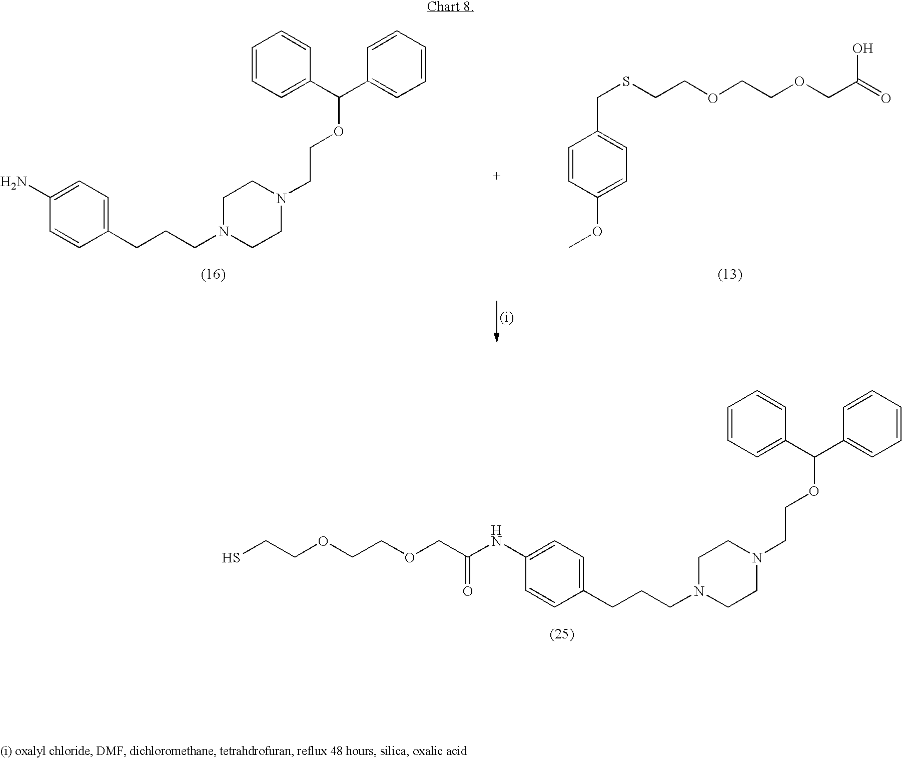

### 2-(2-(2-Chloroethoxy)ethoxy)ethanoic acid (12)

2-(2-(2-chloroethoxy)ethoxy)ethanol (1.69 g, 10 mmols) is dissolved in acetone (50 ml). This solution is added drop wise to a solution of sulfinuric acid (1.5M, 60 ml) containing chromium (VI) oxide (5.79 g, 38 mmols) at 0° C. Upon complete addition of the alcohol the solution is allowed to warm to room temperature for 18 hours. Inorganic chromium salts are removed by filtration and the solution is concentrated under reduced pressure. The crude product is extracted from solution using dichloromethane (3×100 ml) and the combined extracts were dried over magnesium sulfate. After filtration and evaporation under reduced pressure the crude product is obtained as a colorless oil 1.7 g (93%). This is used without further purification.

### 8-(4-Methoxybenzylthio)-3,6-dioxaoctanoic acid (13)

Sodium (0.253 g, 11 mmols) is added to absolute ethanol (50 ml) and stirred at 0° C. for 30 minutes. 4-Methoxy-α-toluenethiol (0.78 ml, 6 mmols) is added and the mixture is stirred at room temperature for 30 minutes. 2-(2-(2-Chloroethoxy)ethoxy)ethanoic acid (1 g, 5.5 mmols) is added and the mixture is heated at reflux for 18 hours. It is cooled to room temperature poured into distilled water (100 ml) and acidified with hydrochloric acid (2M, 1×50 ml). The product is extracted with dichloromethane (2×100 ml) and the organic solution is dried over magnesium sulfate. After filtering the organic solution it is evaporated under reduced pressure. The product is purified using column chromatography on silica eluted with a gradient system running from dichloromethane to dichloromethane 90%: methanol. This gives approximately 1.24 g (94%) of the product as a colorless oil.

### 1-[2-[bisphenylmethoxy]ethyl]-4-(3-(4-aminophenyl)propyl)piperazine (16)

1-[2-[bisphenylmethoxy]ethyl]-4-(3-(4-nitrophenyl)-1-oxopropyl)piperazine (0.9 g, 2.8 mmols) is dissolved in absolute ethanol (10 ml) in a 100 ml round bottomed flask equipped with a stirrer and reflux condenser. Tin (II) chloride dihydrate (2.6 g) is added and the mixture is heated at reflux for 90 minutes. The solution is poured into crushed ice and a solution of sodium carbonate (5%) in water is added until a pH of 8 is obtained. The aqueous solution is extracted with ethyl acetate (3×200 ml) and this is dried over magnesium sulfate. The product is purified using column chromatography on silica gel eluted with ethylacetate 92%: methanol 5%: triethylamine. This gives approximately 0.66 g (78.6%) of the product as a pale yellow oil.

### 3-(4-Chlorophenyl)-8-methyl-8aza-bicyclo[3.2.1]octane-2-carboxylic acid 2-[4-(2-{2-[2-(4-methoxybenzylthio)ethoxy]ethoxy}acetylylamino)phenyl]ethyl ester (17)

8-(4-Methoxybenzylthio)-3,6-dioxaoctanoic acid (0.008 g, 0.027 mmols) is dissolved in dry toluene (10 ml), oxalyl chloride (0.0008 ml is added and then dry dimethyl formamide (1 drop). The mixture is stirred at room temperature for 1 hour and then evaporated to yield crude 8-(4-Methoxybenzylthio)-3,6-dioxaoctonyl chloride (14). The 8-(4-Methoxybenzylthio)-3,6-dioxaoctonyl chloride (14) is dissolved in dry dichloromethane (20 ml), 3β-(p-Chlorophenyl)tropane-2β-carboxylic acid p-aminophenylethyl ester (0.0100 g, 0.025 mmols) and triethylamine (2 drops) are added. The mixture is heated at reflux for 18 hours cooled and evaporated under reduced pressure. (17) is purified using column chromatography on silica gel eluted with ethyl acetate 98%: triethyl amine. This yields approximately 0.006 g (34%) of (17) as a tar.

### 3-(4-Chlorophenyl)-8-methyl-8-azabicyclo[3.2.1]octane-2-carboxylic acid 2-(4-{2-[2-(2-mercaptoethoxy)ethoxy]acetylamino}phenyl)ethyl ester (18)

3-(4-Chlorophenyl)-8-methyl-8aza-bicyclo[3.2.1]octane-2-carboxylic acid 2-[4-(2-{2-[2-(4-methoxybenzylthio)ethoxy]ethoxy} acetylylamino)phenyl]ethyl ester (0.021 g, 0.03 mmols) is dissolved in trifluoroacetic acid (5 ml) and cooled to 0° C. Anisole (0.05 ml) and mercury (II) acetate (0.011 g, 0.036 mmols) are added to this solution and it is stirred at 0° C. for 2 hours. The solvent is evaporated, the product is triturated with diethyl ether and vacuum dried. Then it is dissolved in glacial acetic acid (10 ml) and hydrogen sulfide is bubbled through the solution for 30 minutes. The solution is filtered evaporated and methanolic hydrogen chloride (10 ml) is added to the tar. Then it is evaporated under reduced pressure and this procedure is repeated 5 more times. After drying under vacuum 0.008 g (44%) of (17) is obtained as the hydrochloride salt.

### Diphenylmethanol (19)

This compound was synthesised using the method reported in the Journal of the Chemical Society, 1960, 2133, by Mole. A 1 litre three necked round bottomed flask equipped with a stirrer a reflux condenser and a 200 ml pressure equalising addition funnel, is charged with magnesium turnings (15.36 g, 630 mmols). Dry diethyl ether (150 ml) and iodine (0.1 g, 0.3 mmols) are added. The mixture is heated at reflux until the purple iodine colour disappeared and to this solution was added 5 ml of a solution of bromo benzene (65.2 ml, 97.24 g, 620 mmols) in 150 ml of anhydrous ether. The reaction mixture is heated at reflux until a cloudy grey color forms. The heat is removed and the remaining bromo benzene is added drop wise at such a rate so as to maintain reflux. The solution is heated at reflux for a further hour after the addition of bromobenzene is complete. After which it is cooled to 10° C. in an ice acetone bath and benzaldehyde (60 ml, 62.4 g, 588 mmols) in anhydrous ether 200 ml is added drop wise so that the temperature of the reaction mixture does not exceed 20° C. The reaction mixture is allowed to warm to room temperature after the addition of benzaldehyde and it is stirred at room temperature for a further 18 hours. The reaction is quenched by adding ammonium chloride solution (100 ml) at 0° C. After which the organic layer is separated washed with water and dried over magnesium sulfate. The solution is filtered and evaporated and the product is washed with hexanes (150 ml) to give approximately 51.6 g (45%) of the product as a colorless solid mpt=64-64.5° C.

### 1,1′-[(2-Chloroethoxy)methylene]bis-benzene (20)

This compound was synthesized using the method reported in the European Journal of Medicinal Chemistry, 1980, 15 (4), 363, by Van Der Zee P. et. al. Freshly distilled 2-chloroethanol (11 g) is added to toluene (25 ml) in a 500 ml round bottomed flask, equipped with a reflux condenser a 200 ml pressure equalizing funnel and a stirrer. Concentrated sulphuric acid (1 ml) is added and the mixture is heated at reflux for 5 hours during which Diphenylmethanol (11,56 g, 90 mmols) dissolved in toluene (150 ml) is added drop wise. Then the solution is cooled to room temperature the aqueous layer is separated and the organic solution is washed with sodium bicarbonate (sat, 100 ml) and water (2×100 ml). It is dried over magnesium sulphate filtered and evaporated. The product is purified by vacuum distillation and this gives approximately 6 g (26%) of the product as a colorless oil.

### 1-[2-[bisphenylmethoxy]ethyl]piperazine (21)

Piperazine hexahydrate (47 g, 240 mmols) is added to toluene (100 ml) and anhydrous potassium carbonate (66 g, 600 mmoles) is added. The mixture is heated at reflux and 1,1′-[(2-Chloroethoxy)methylene]bis-benzene (20 g, 80 mmols) is added drop wise over five hours. After refluxing for a further 18 hours the solution is allowed to cool to 70° C. washed with water (5×250 ml), dried over magnesium sulfate, filtered and evaporated. The resulting yellow oil is converted to a dimaliate salt by crystallising from diethyl ether. This gives approximately 25 g (50%) of the product as a colourless solid.

### Para-Nitrohydrocinnamic Acid (22)

This compound is prepared using the method described by Moloney in the journal of medicinal chemistry, 1999, volume 42 No 14 page 2504. Hyrocinnamic acid is added to concentrated sulfuric acid (49 ml) in a 3 necked 250 ml round bottomed flask equipped with a stirrer and thermometer. The flask is cooled to 0° C. in an ice bath and concentrated nitric acid (10 ml) is added drop wise maintaining the temperature below 10° C. The solution is stirred for a further hour at 0° C. after the complete addition of the nitric acid. Then the ice bath is removed and the mixture is stirred at room temperature for 30 minutes. The resulting orange solution is poured into ice and the crude product is collected by filtration. The product is air dried and re-crystallised from ethyl acetate giving approximately 10 g (29%) of the para-nitrohyrocinnamic acid as a colorless solid.

### 1-[2-[bisphenylmethoxy]ethyl]-4-(3-(4-nitrophenyl)-1-oxopropyl)piperazine (23)

para-Nitrohyrocinnamic acid (2.8 g, 9.5 mmols) is added to dry toluene (10 ml), in a 250 ml round bottomed flask equipped with a stirrer and a reflux condenser. Oxalyl chloride (1 ml) is added, after which a catalytic quantity of dry DMF (2 drops) is also added and the mixture is stirred at room temperature for 2 hours. The solvent is removed by evaporation and the crude acid chloride is dissolved in dry dichloromethane (100 ml). Dry triethylamine (10 ml) and 1-[2-[bisphenylmethoxy]ethyl]piperazine (1.84 g, 9.5 mmols) are dissolved in dry dichloromethane (50 ml) and added to the solution of p-nitrohyrdrocinnamyl chloride. The mixture is heated at reflux for 18 hours under argon in a 250 ml round bottomed flask equipped with a stirrer and reflux condenser. The solvent is removed under reduced pressure and the product is purified using silica gel chromatography eluted with dichloromethane 96%: methanolic ammonia. The resulting yellow oil which is converted into the yellow maleate salt by crystallisation from diethyl ether. This gives approximately 4.3 g (99%) of the product.

### 1-[2-[bisphenylmethoxy]ethyl]-4-(3-(4-nitrophenyl)propyl)piperazine (24)

1-[2-[bisphenylmethoxy]ethyl]-4-(3-(4-nitrophenyl)-1-oxopropyl)piperazine (5 g, 14.8 mmols) in a 250 ml round bottomed flask equipped with a stirrer and a reflux condenser is dissolved in dry THF (100 ml). Alane in toluene (0.5M, 59 ml) is added and stirred at room temperature for 30 minutes. The reaction is quenched with sodium hydroxide solution (10%, 200 ml). The aqueous solution is extracted with diethyl ether (3×150 ml), dried over magnesium sulfate filtered and evaporated. The product is purified using silica gel chromatography eluted with a gradient system eluted with ethyl acetate 90%: methanol to ethyl acetate 87%: methanol 10%: triethylamine. This gives approximately 3.35 g (68%) of the product as a pale yellow oil.

### N-(4-(3-[4-(2-Benhydryloxyethyl)piperazine-1-yl]propyl)phenyl-2-[2-(2-mercaptoethoxy)ethoxy]acetamide (25)

8-(4-Methoxybenzylthio)-3,6-dioxaoctanoic acid (0.6 g, 2.2 mmols) is dissolved in dry toluene (50 ml) under nitrogen in a 100 ml round bottomed flask equipped with a stirrer and a reflux condenser. Oxalyl chloride (0.5 ml) and a catalytic quantity of dimethyl formamide (1 drop) are added. The solution is stirred at room temperature for 2 hours, then evaporated under reduced pressure. The resulting crude 8-(4-Methoxybenzylthio)-3,6-dioxaoctonyl chloride (14) is dissolved in dry dichloromethane (100 ml) in a 250 ml round bottomed flask equipped with a reflux condenser and a stirrer. 1-[2-[bisphenylmethoxy]ethyl]-4-(3-(4-aminophenyl)propyl)piperazine (0.66 g, 2.2 mmols) in dry tetrahydrofuran and triethylamine (5 ml) are added and the mixture is allowed to reflux under nitrogen. Then the solvent is evaporated and the product is columned on silica eluted with ethyl acetate 93%: methanol 5%: triethylamine. The crude product is converted to the oxylate salt by dissolving it in methanol (50 ml) and adding oxalic acid (1 g) dissolved in methanol (20 ml). The resulting solid is left standing at room temperature for 18 hours and removed by filtration. The oxalate salt is converted back to the base and the product is purified by column chromatography on silica eluted with a gradient system running from dichloromethane (90%): methanol to dichloromethane (87%): methanol 10%: triethylamine. This gives the product as a yellow oil it is converted back to the oxylate salt, as described above and this is filtered and air dried. To yield approximately 0.28 g (28.9%) of the product as a yellow solid.

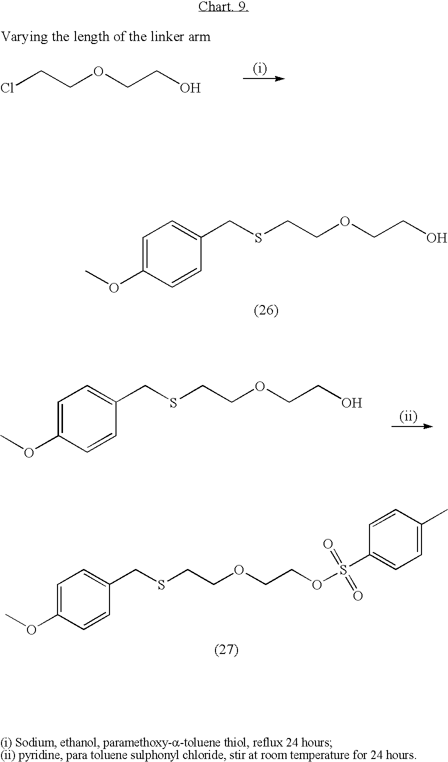

The length of the linker arms of the present invention may be changed. Accordingly, at least a di and tetra polyethylene glycol linker arm may be synthesised. The synthetic routes for these compounds are outlined in charts 9 and 10, below. The linker arm is shortened in chart 9 and lengthened in chart 10.

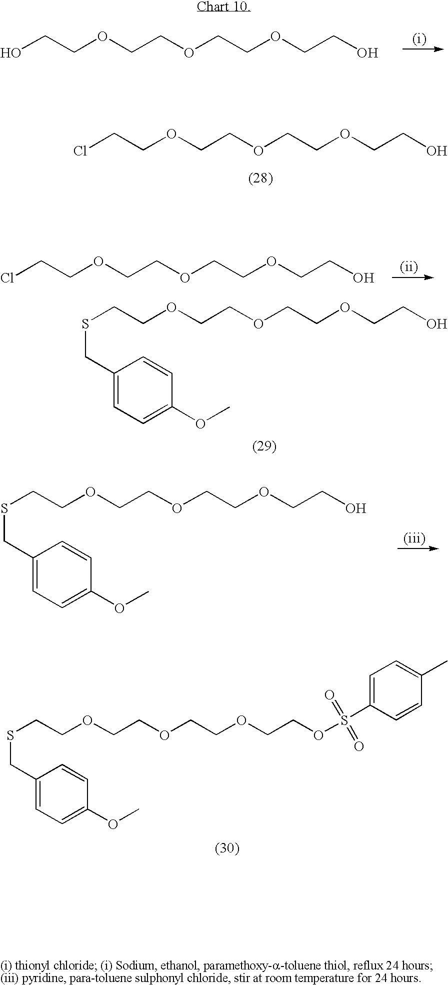

### 5-(4-methoxybenzthio)-3-oxapentanol (26)

Sodium metal (0.92 g, 40 mmols) is added to absolute ethanol (100 ml) at 0° C. 4-methoxy-α-toluenethiol (5.6 ml) is added after the sodium has completed reacting. 2-(2-chloroethoxy)ethanol (5.48 g, 44 mmols) is added 30 minutes later and the mixture is heated at reflux for 18 hours. The solution is cooled to room temperature and added to saturated ammonium chloride solution (100 ml). It is extracted into dichloromethane (3×100 ml). After drying the combined organic extracts over magnesium sulfate and filtering, the dichloromethane is removed under reduced pressure. The product is purified by column chromatography on silica gel eluted with a gradient system from dichloromethane to dichloromethane 90%: methanol. This yields approximately 5.9 g (60%) of the product as a colorless oil.

### 5-(4-Methoxybenzylthio)-3-oxapentyl tosylate (27)

5-(4-Methoxybenzylthio)-3-oxapentanol (26), (2.42 g, 10 mmols) is added to dry pyridine (10 ml) and cooled to 0° C., para-toluene sulfonyl chloride (2.59 g, 14 mmols) is added and the mixture is allowed to warm to room temperature over an 18 hour period with stirring. Water (50 ml) and dichloromethane (100 ml) are added. The organic layer is separated, it is washed with hydrochloric acid (2M, 1×100 ml) and water (50 ml). The organic solution is dried over magnesium sulfate, filtered and evaporated. The product is purified using column chromatography on a silica column eluted with a gradient system from petroleum spirit 70%: diethyl ether to petroleum spirit 30%: diethyl ether. This yields approximately 0.13 g (3.6%) of the product as a colorless oil.

### 2-(2-(2-(2-Chloroethoxy)ethoxy)ethoxy)ethanol (28)

Tetraethylene glycol (192 g, 990 mmols) is added to dry chloroform (200 ml) in a 1 L flask equipped with a stirrer, reflux condenser and a thermometer. Dry pyridine (80 ml) is added to this solution and it is cooled to 0° C. Freshly distilled thionyl chloride (73 ml) is added over a 4 hour period, whilst maintaining the temperature below 110° C. After all the thionyl chloride has been added the solution is heated at reflux for 18 hours. Then the chloroform is removed under reduced pressure and the resulting residue is extracted with water (2×100 ml). The aqueous solution is washed with hexane's (2×100 ml) and the crude product is extracted into toluene (5×100 ml). Then the solvent is dried with magnesium sulfate filtered and evaporated. The product is purified by distillation under reduced pressure using an aspirator (Bpt=140-160° C.). This yields approximately 19.8 g (9.4%) of the product as a colorless oil.

### 11-(4-Methoxybenzylthio)-3,6,9-trioxaundecanol (29)

Sodium metal (0.8 g) is added to absolute ethanol (100 ml) at 0° C. in a 250 ml round bottomed flask equipped with a stirrer and a reflux condenser. After the sodium has completely reacted with the ethanol 4-methoxy-α-toluenethiol (4.9 ml) is added. This is stirred at room temperature for 30 minutes. Then 2-(2-(2-(2-Chloroethoxy)ethoxy)ethoxy)ethanol (7.4 g, 34 mmols) is added and the mixture is heated at reflux for 18 hours. After cooling to room temperature it is added to saturated ammonium carbonate solution (100 ml) and extracted into dichloromethane (3×100 ml). The combined organic extracts were dried over magnesium sulfate filtered and evaporated. The product is purified by column chromatography on silica gel eluted with a gradient system from dichloromethane to dichloromethane 90%: methanol. This yields approximately 6 g (53%) of the product as an oil.

### 11-(4-Methoxybenzylthio)-3,6,9-trioxaundecanyl tosylate (30)

2-(2-(2-(2-Chloroethoxy)ethoxy)ethoxy)ethanol (5.9 g, 18 mmols) is dissolved in dry pyridine (10 ml) and cooled to 0° C., in a 50 ml round bottomed flask equipped with a stirrer and a calcium chloride drying tube. Para-toluene sulfonyl chloride (4.66 g, 24 mmols) is added to the mixture and it is stirred for 18 hours, during this period the temperature of the reaction mixture is allowed to increase from 0° C. to room temperature. Water (50 ml) is added to the reaction mixture and it is extracted with dichloromethane (2×50 ml). The combined organic extracts are washed with hydrochloric acid (2M, 2×50 ml), saturated sodium bicarbonate solution (2×50 ml) and water (2×50 ml). After which the solution is dried over magnesium sulfate, filtered and evaporated. The product is purified using column chromatography on a gradient system from ethyl acetate 40%: hexane to ethyl acetate. This yields approximately 6.7 g (77.8%) of the product as a colorless oil.

### Linker Arm Synthesis with Other Functionalities

The linker arms of the present invention may be synthesized with other functionalities, including a chloride and an amine functionality. The synthesis of these compounds is outlined in chart 11, below. The amino functionality may also be attached to drugs or biologically active molecules such as cholesterol, proteins and antibodies, via an amide linker.

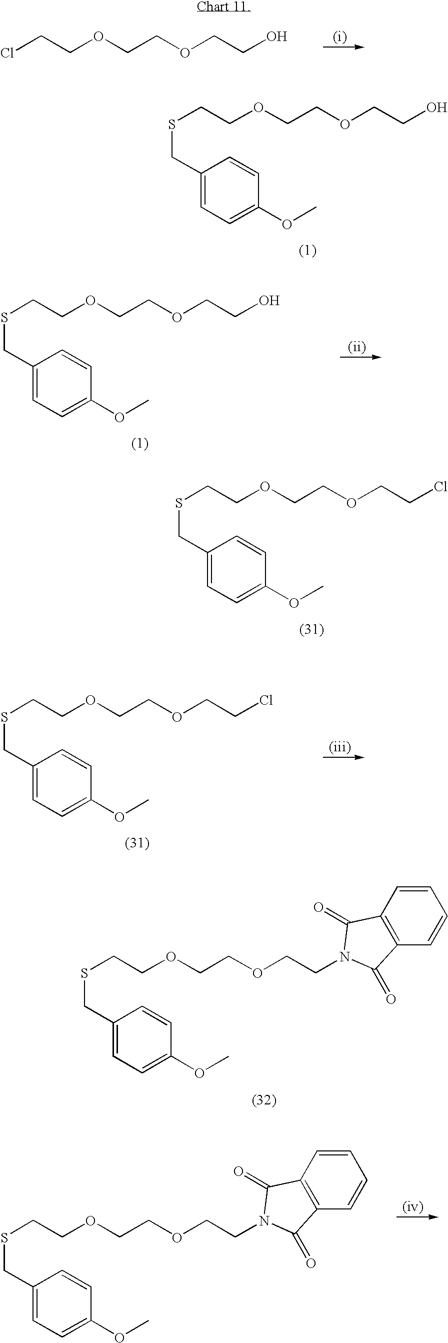

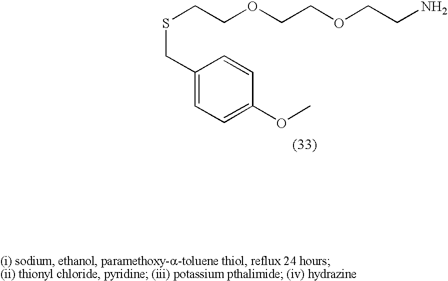

### 8-(4-Methoxybenzylthio)-3,6-dioxaoctyl chloride (31)

8-(4-Methoxybenzylthio)-3,6-dioxaoctanol (1.72 g, 6 mmols) is dissolved in dry dichloromethane (30 ml) and dry pyridine (0.97 ml, 12 mmols) is added. The solution is stirred for 5 minutes then thionyl chloride (0.5 ml) is added. The mixture is heated at reflux overnight. Then it is poured into hydrochloric acid (2M, 25 ml) and the organic layer is separated. The aqueous solution is extracted with dichloromethane (2×25 ml) and the combined organic extracts are washed with water (10 ml). After drying over magnesium sulfate the solution is filtered and evaporated. The product is purified using silica gel chromatography eluted with a gradient system from petroleum ether 60%: diethyl ether to petroleum ether 40%: diethyl ether. This yields approximately 0.39 g (57%) of the product as a colorless oil.

### 8-(4-Methoxybenzylthio)-1-(N-phthalimido)-3,6-dioxaoctane (32)

8-(4-Methoxybenzylthio)-3,6-dioxaoctyl chloride (0.53 g, 1.7 mmols) is dissolved in dimethyl formamide and potassium phthalimide (0.32 g, 2 mmols) is added. The mixture is heated at 100° C. for 18 hours and then cooled to room temperature. It is poured into water (100 ml) and extracted with diethyl ether (3×100 ml), after drying over magnesium sulfate it is filtered and evaporated. The product is purified using silica gel chromatography eluted with a gradient system from petroleum ether 60%: diethyl ether to petroleum ether 40%: diethyl ether. This gave 0.39 g (57%) of the product as a colorless oil.

### 8-(4-Methoxybenzylthio)-3,6-dioxaoctylamine (33)

8-(4-Methoxybenzylthio)-1-(N-phthalimido)-3,6-dioxaoctane (0.39 g, 0.96 mmols) is dissolved in absolute ethanol and hydrazine hydrate (1 ml) is added. The mixture is heated at reflux for 1 hour and the solvent is removed under reduced pressure. Water (10 ml) and sodium hydroxide solution (1M, 10 ml) are added to the resulting tar and the product is extracted with diethyl ether (3×50 ml). The ethereal solution is dried over magnesium sulfate filtered and evaporated to yield approximately 0.25 g (92%) of the product as an oil.

### Attachment of Biologically Active Compounds to the Linker Arm

A biologically active organic compound may be attached to the linker arm as follows: 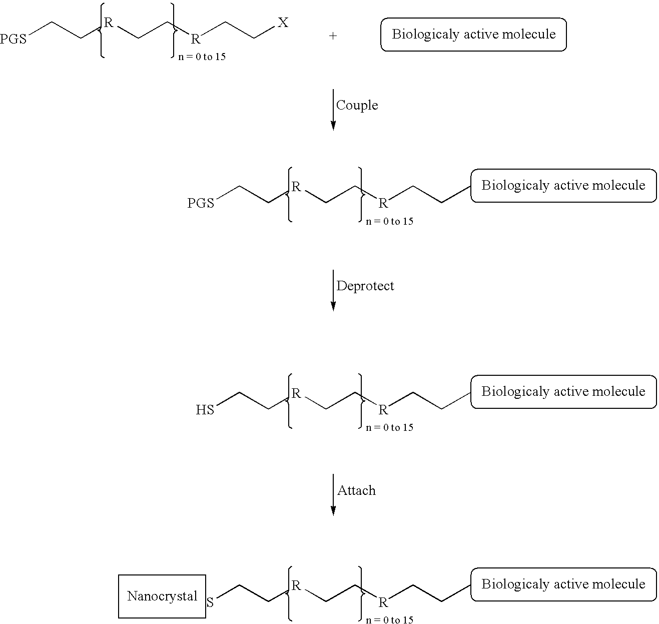
Where X is Cl, Br, I, OTs, OMs, OTf, NH₂, SH, OH, CisO, COCl, CO₂H, etc. The biologically active molecule is attached to the linker arm via a functional group or a methylene group. R may be O, NH, S, CH₂, etc. PG is a protecting group and may be para-methoxy benzyl, benzyl, a thioamide, a thio ether, etc.

### Attaching Linker Arms to Nanocrystal Core Shells

This example discloses a method of attaching linker arms of the present invention to nanocrystal core shells. An example of the methodology used is outlined below:

9 mg of trioctylphosphine oxide coated core shells are weighed out and suspended in pyridine (2 ml). The concentration and thus the number of moles of nanocrystals may be determined before hand using UV-vis spectroscopy. This suspension is stirred at 60° C. for 24 hours, N-(4-(3-[4-(2-Benhydryloxyethyl)piperazine-1-yl]propyl)phenyl-2-[2-(2-mercaptoetoxy)ethoxy] acetamide (25), (100 mg) is dissolved in dichloromethane (100 ml) and 2.7 ml of this solution is added to the solution of nanocrystals. This gives approximately 100 ligands per core shell. The solution is stirred at 60° C. under argon for 2 hours. Upon cooling to room temperature the solution is added to hexanes. Ligand coated core shells crystallise out of solution and are collected by filtration.

The water solubility of the ligand functionalised core shells may be increased if necessary by using a modification of the method of Fred Mikulec (private communication). Mercaptoacetic acid (1 ml) and dimethyl formamide (1 ml) are added to the ligand coated core shells and stirred at room temperature under argon for 2 hours. After cooling to room temperature the solution is diluted with dimethyl formamide (100 ml) and potassium teriary butoxide (1.61 g) is added. The resulting solid is collected by centrifugation and is washed with tetrahydrofuran (4×100 ml) and methanol (7×100 ml). The product is collected by centrifugation to yield 45 mg of 1-[2-bisphenylmethoxy]ethyl]-4-(3-(4-(3,6-dioxa-8-thiol)octanamidophenyl)propyl piperazine (25) coated nanocrystals. After drying the precipitate under reduced pressure for 4 days at room temperature the ligand coated cores can be dissolved in a minimum quantity of buffer in a pH range of 6 to 8.

A variety of amphiphilic coatings have been developed one such amphiphilic coating is based upon modified amphiphilic poly (acrylic acid) polymer. When nanocrystals (Quantum Dots) are coated with an amphiphilic Poly(acrylic acid) polymer they are called AMP™ quantum dots. These dots may be pegilated and functionalized in the same manner.

The ligands described in this patent may be attached to the pegilated AMP dots via a variety of different strategies. One such method utilizes the bromo intermediates that are intermediates in the thiol synthesis. These bromo intermediates may be covalently attached to the end of the peg chain using phase transfer catalysis. Phase transfer coupling using a tertiary butyl ammonium bromide catalyst yields derivitised materials that have different electrophoretic motilities from the pegilated AMP dots. The phase transfer coupling of biologically active molecules to the reactive group X at the end of the PEG chain is shown in Schemes A and B.

In the phase transfer coupling process the biologically active ligand is dissolved in an organic solvent such as ether or methylene chloride. The water soluble pegilated AMP quantum dots are dissolved in water or buffer the catalyst is added and the two immiscible solutions are stirred together for 30 minutes. The organic solvent is then removed and the derivatized dots are purified via column chromatography on sephadex.

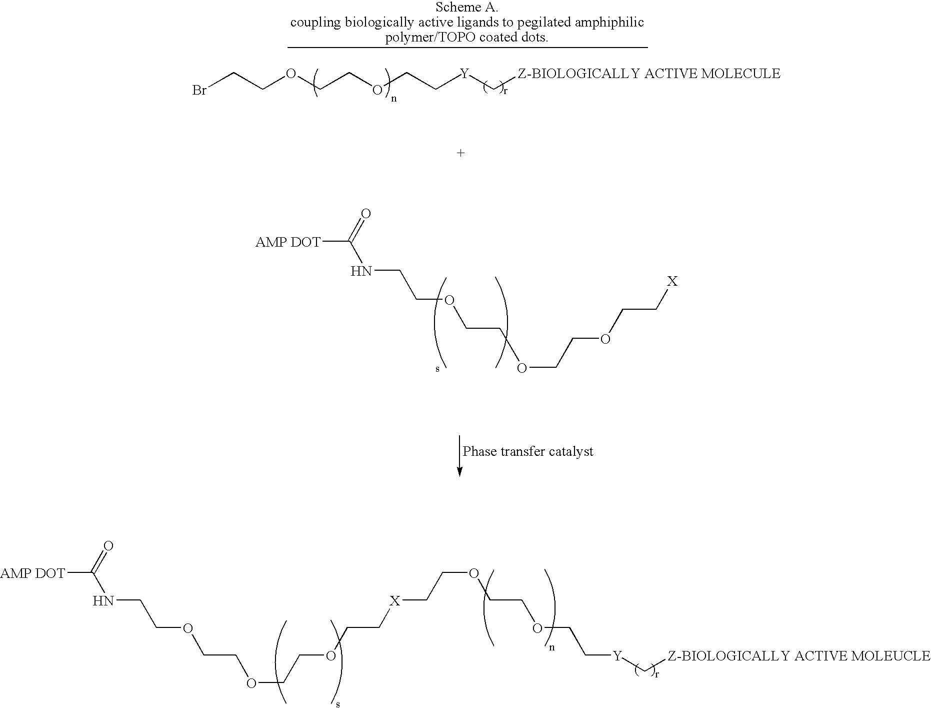

X in Scheme A refers to a reactive group such as OH, NH₂, or SH; s has a value of 10 or greater and in preferably n is 21, 22, 23, 24, 25, 26, 27, 28, 29 and 30. n has a value of 2 or greater. Y is O, S, NH, CH₂, CONH, NHCO, NH, SO, SO₂NH, NHSO₂, carbamate, thiocarbamate, NHR (Where R is aryl or alkyl); r has a value of either 0 or a value between 2 and 15, Z is the point of attachment to a biologically active molecule and may be O, S, NH, CH₂, CONH, NHCO, NH, SO, SO₂NH, NHSO₂, carbamate, thiocarbamate, NHR (Where R is aryl or alkyl). The biologically active molecule may be a drug or neurotransmitter.

Biologically active ligands that may be attached to pegilated AMP dots claimed in this patent include the following; 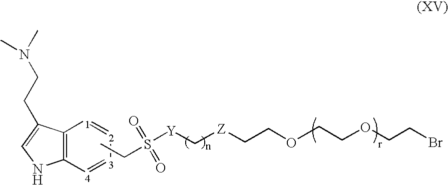
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 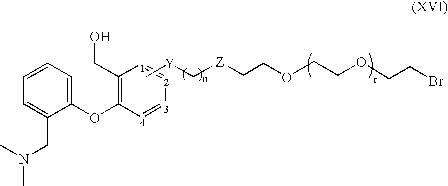
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 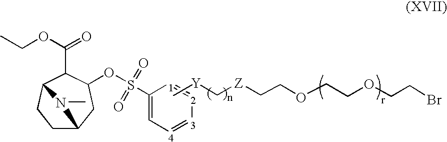
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1, 2 or 3. Preferably position 3. 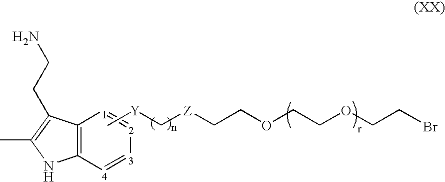
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 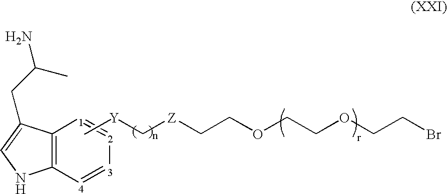
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 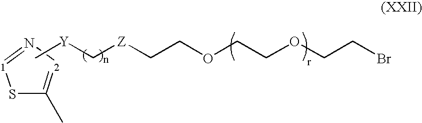
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1 or 2. Preferably position 2. 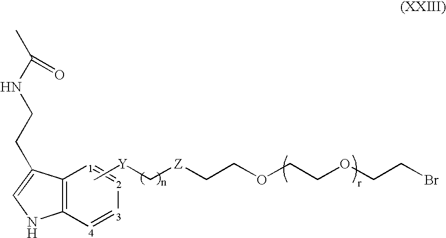
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1,2, 3 or 4. Preferably position 2. 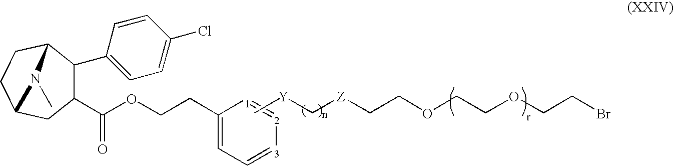
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). The linker arm may be attached to positions 1, 2 or 3. Preferably position 3. 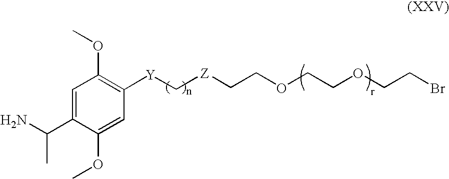
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl). 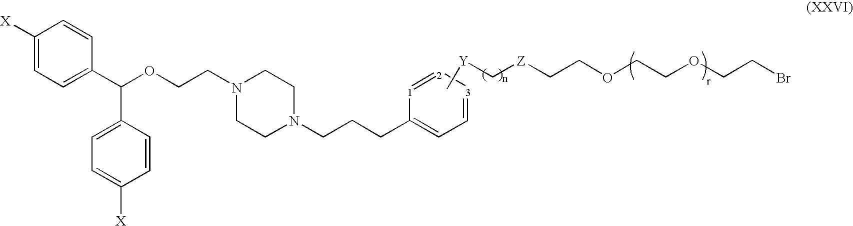
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl).

The linker arm may be attached to positions 1, 2 or 3. Preferably position 3. 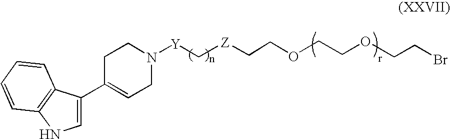
r is 1-10. In certain embodiments, r is 2, 3, 4, or 5. Z is O, S, NH, CH₂, CONH, NHCO, NH, SO, SO₂NH, NHSO₂, carbamate, thiocarbamate, NHR(R is aryl or alkyl).
n is 1-15. In certain embodiments, n is 4,5,6,7,8,9, or 10. Y is O, S, NH, CH₂, CONH, NHCO, NH, SO, SO₂NH, NHSO₂, carbamate, thiocarbamate, NHR(R is aryl or alkyl).

The poly ethylene glycol chain attached to the alkyl spacer shown in Scheme A may be replaced with an alkyl chain that is terminated with a displaceable group such as bromo, iodo, chloro, tosyl, etc. This may be coupled up to the pegilated dots using the same phase transfer conditions previously described. Thus the alkyl analogues of compounds (XV) to (XXVII) in which the alkyl chain is terminated with a tosyl, bromo, chloro, or iodo functionality can be directly attached to pegilated AMP quantum dot the methodology is outlined in Scheme B.

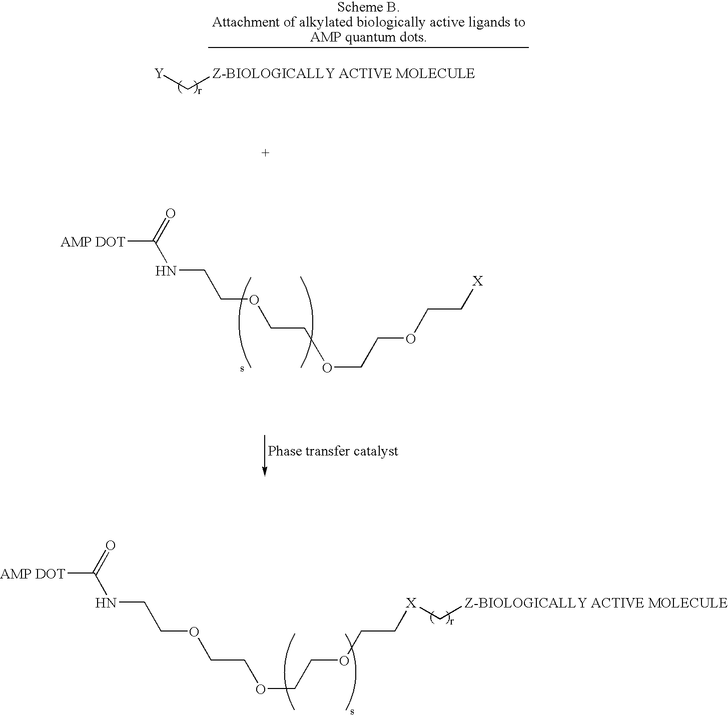

The biologically active molecule in Scheme B may be a drug or neurotransmitter. In particular the biologically active substances that are alkylated derivatives of compounds (XV) through to (XXVII) are claimed. The length of the alkyl chain may be varied and r is 1-15, Z is the point of attachment to the drug or neurotransmitter and may be one of the following functionalities; NH, O, S, CH₂, NH, NHR, CONH, NHCO, SO, SO₂NH, NHSO₂, Carbamate, and thio carbamate. R is either alkyl or aryl; Y may be one of the following I, Br, Cl, or tosyl; X may be NH₂, OH, or SH; s is 10 ethylene oxide units or greater.

In addition to coupling biologically active ligand to pegilated AMP quantum dots compounds (I) through (XIV) may also be conjugated to these nanocrystals using maleimide coupling chemistry. The synthetic methodology of this chemistry is outlined in Scheme C.

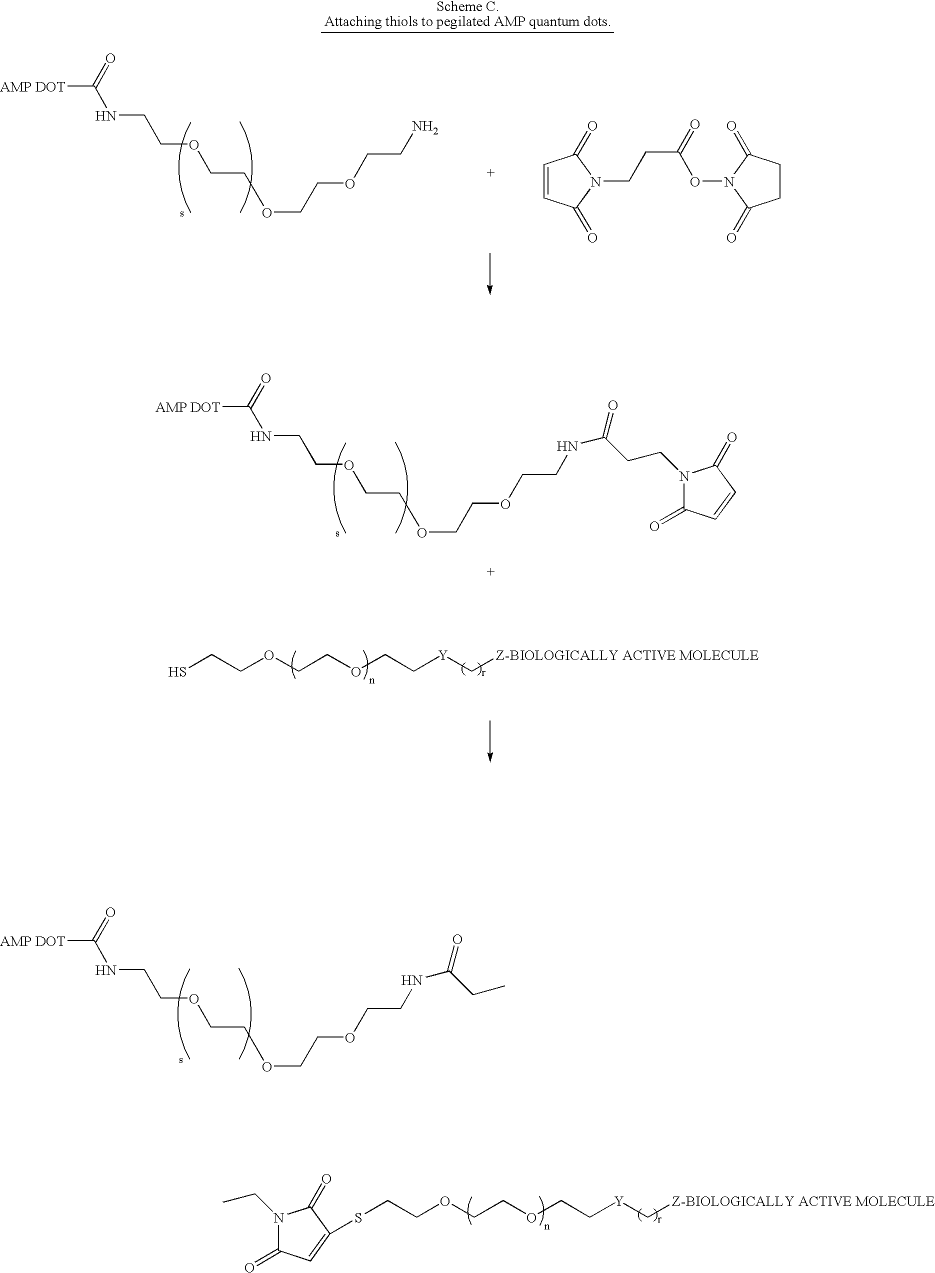

Scheme C shows the methodology for attaching biologically active ligands to amino terminated PEGs; s has a value of 10 or greater and n has a value of 2 or greater. Y is 0, CONH, S, NH, CH₂, NR, where R is an alkyl chain or an aromatic ring; r has a value of either 0 or a value between 2 and 15, Z is the point of attachment to the drug or neurotransmitter molecule and may be O, NH, NR, S, CONH, CH₂, SO, NHCO, SO₂NH, NHSO₂, cabamate, thiocarbamate; where R is an alkyl chain or an aromatic ring. The biologically active molecule may be any one of compounds (I) through (XIV) or other drugs, neurotransmitters, hormones, peptides, proteins, nucleic acids, antibodies or any other biologically active material. A different spacer is used to couple compounds (I) through (XIV) to pegilated AMP quantum dots when the PEG chain is terminated with an alcohol group. The method used is outlined in Scheme D. 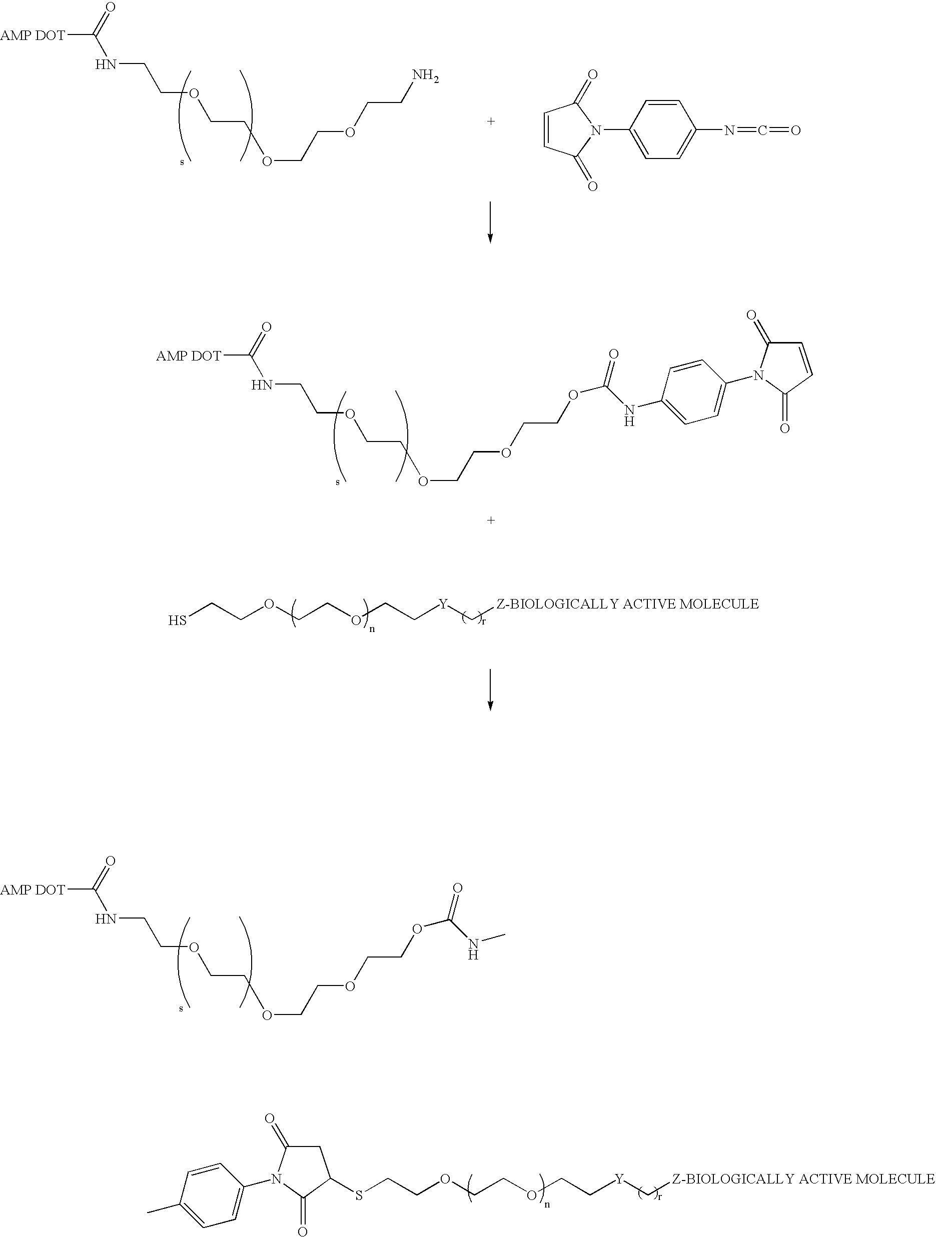
s has a value of 10 or greater and n has a value of 2 or greater. Y is O, CONH, S, NH, CH₂, NR, where R is an alkyl chain or an aromatic ring; r has a value of either 0 or a value between 2 and 15, Z is the point of attachment to the drug or neurotransmitter molecule and may be O, NH, NR, S, CONH, CH₂, SO, NHCO, SO₂NH, NHSO₂, cabamate, thiocarbamate; where R is an alkyl chain or an aromatic ring. The biologically active molecule may be any one of compounds (I) through (XIV) or other drugs and neurotransmitters.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification and Example be considered as exemplary only, and not intended to limit the scope and spirit of the invention.

Unless otherwise indicated, all numbers expressing quantities of ingredients, properties, amounts, and so forth used in the Specification and claims are to be understood as being modified in all instances by the term “about.” Accordingly, unless indicated by the contrary, the numerical parameters set forth in the Specification and claims are approximations that may vary depending upon the desired properties sought to be determined by the present invention.

Notwithstanding that the numerical ranges and parameters setting forth the broad scope of the invention are approximations, the numerical values set forth in the experimental sections or the example sections are reported as precisely as possible. Any numerical value, however, inherently contain certain errors necessarily resulting from the standard deviation found in their respective testing measurements.

Throughout this application, various publications are referenced. The disclosures of these publications, and the references cited therein, including those listed below, in their entireties, are hereby incorporated by reference in their entirety into this application in order to more fully describe the state of the art to which this invention pertains.

### REFERENCES

1. Xiaohu Gao, Yuanyuan Cui, Richard M Levenson, Leland W K Chung, Shuming Nie, Nature Biotechnology, 22, 969-976, 2004
2. Melissa A. Petruska, Andrew P. Bartko, VictorI. Klimov, Journal of the American Chemical Society, 126, 714-715, 2004
3. Ming Zheng, Zhigang Li, Xueying Huang, Langmuir, 20, 4226-4235, 2004 Byron Ballou, B. Christoffer Lagerholm, Lauren A. Ernst, Marcel P. Bruchez, Alan S. Waggoner, 15, 79-86, 2004

1. A nanocrystal compound comprising:
a nanocrystal, and attached thereto a compound of the following formula: 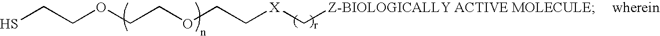
n is 0 or an integer from 1 to 48;
X and Z are independently O, NH, N—R, S, CH₂, CO, COHN, NHCO, SO, SO₂NH, NHSO₂, carbamate and thio carbamate;
R is alkyl or aryl;
r is 0 or an integer from 1 to 15; and
wherein s is the attachment point to a nanocrystal compound.

2. The nanocrystal compound of claim 1, wherein the biologically active molecule is selected from the group consisting of serotonin, serotonin, cocaine, phenyl tropane, phenylisopropylamine, dopamine, chlormethaizole, RTI-4229-75, GBR 12935, RTI-4229-75, GBR 12935; and derivatives thereof.

3. The nanocrystal compound of claim 1, wherein the biologically active molecule is a CNS drug.

4. The nanocrystal compound of claim 1, wherein the compound is of the following formula: 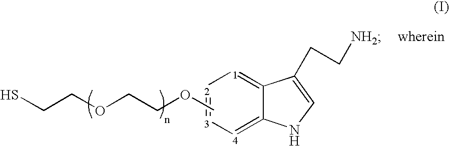
n is 0-10.

5. The nanocrystal compound of claim 4, wherein n is 2, 3, 4, or 5.

6. The nanocrystal compound of claim 1, wherein the compound is of the following formula: 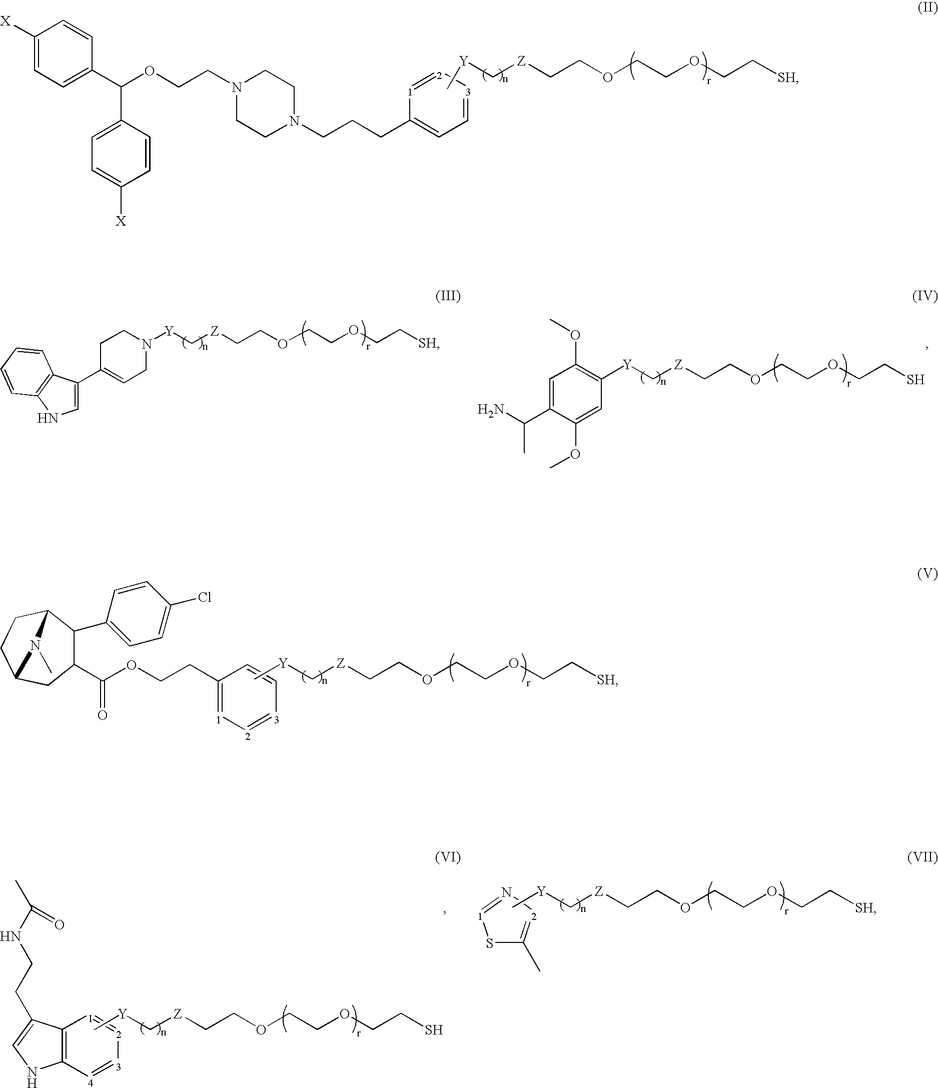 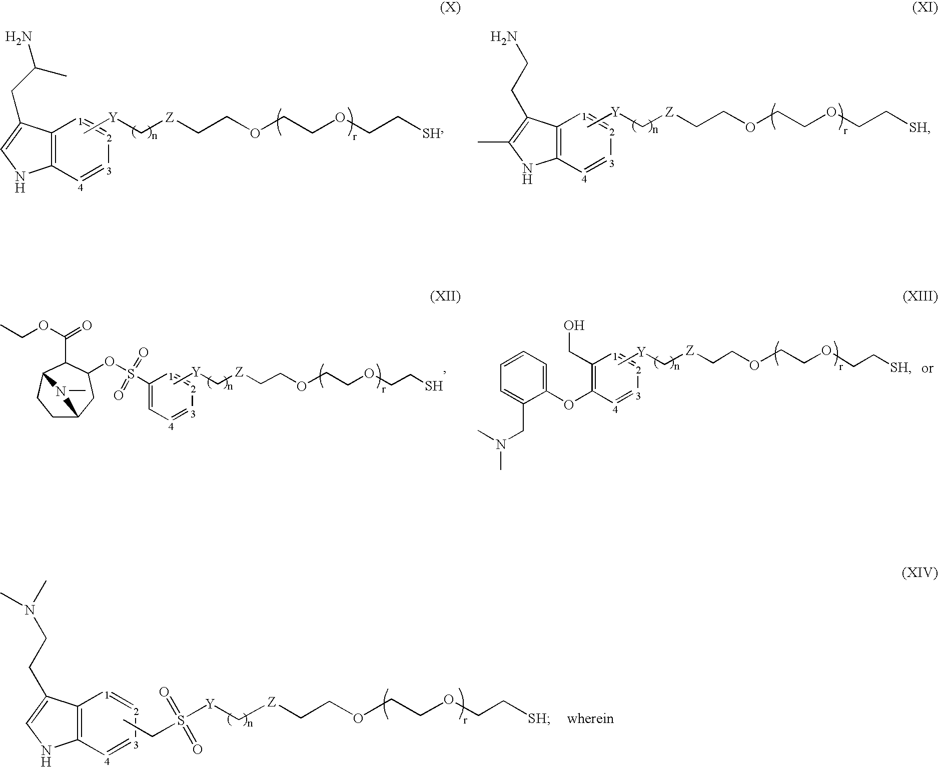
r is 0-10;
Z, Y are independently O, S, NH, CH₂, CONH, NHCO, NH, SO, SO₂NH, NHSO₂, carbamate, thiocarbamate, NH—R;
R is aryl or alky; and
n=1-15.

7. The nanocrystal compound of claim 6, wherein r is 2, 3, 4, or 5; and n is 4,5,6,7,8,9, or 10.

8. The nanocrystal compound of claim 1, wherein the compound is:

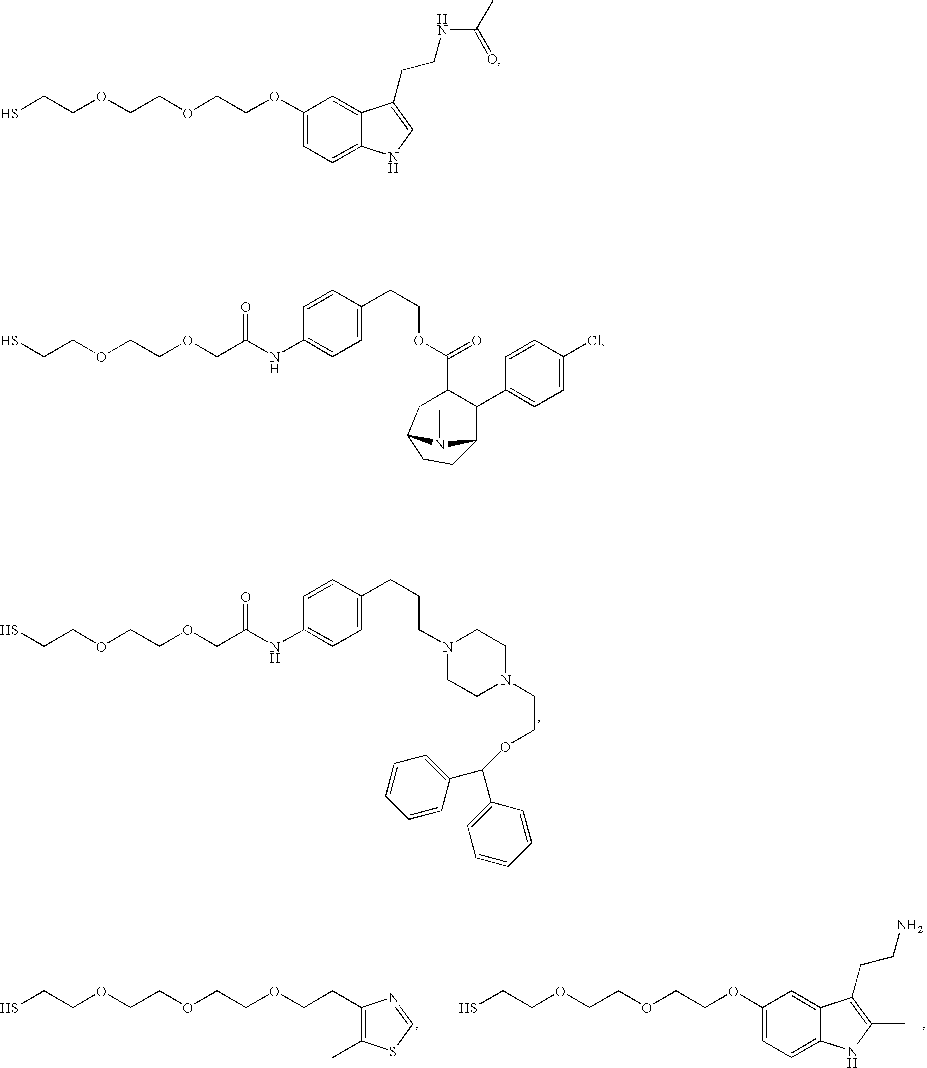

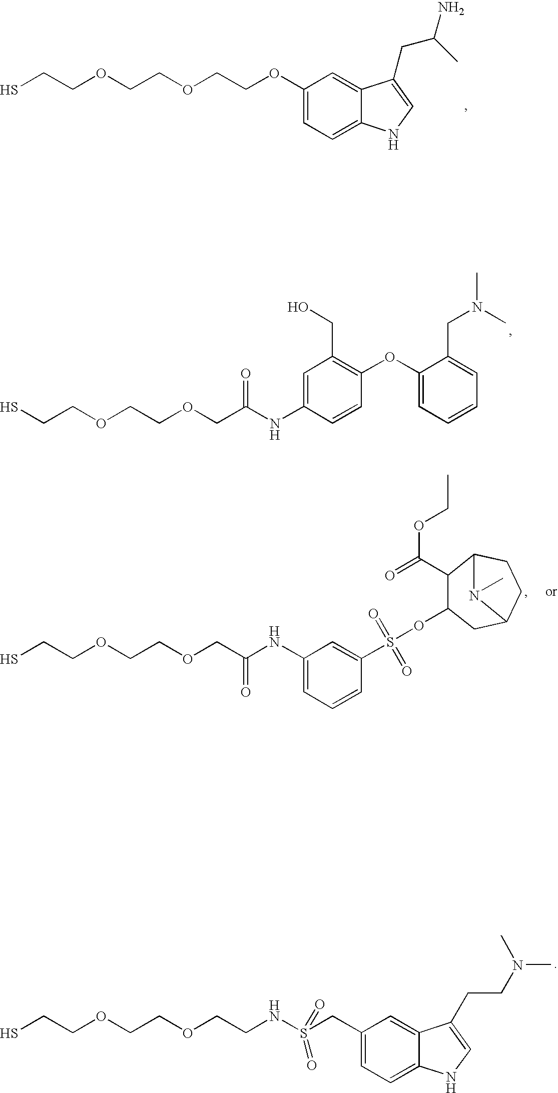

9. The nanocrystal compound of claim 1, wherein the nanocrystal is a CdSe core/ZnS shell nanocrystal.

10. A nanocrystal compound comprising:
a pegilated AMP nanocrystal, and attached thereto a compound of the following formula: 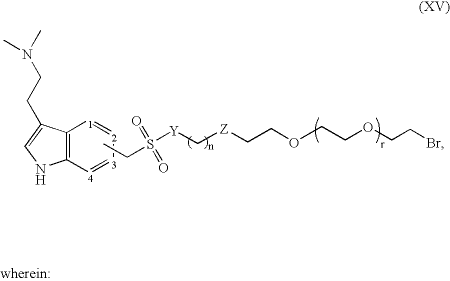
Z and Y are independently O, S, NH, CH₂, CONH, NHCO, NH, SO, SO₂NH, NHSO₂, carbamate, thiocarbamate, NH—R;
R is aryl or alkyl;
n is 1-15; and
r is 1-10.

11. The nanocrystal compound of claim 10, wherein n is 4,5,6,7,8,9, or 10.

12. The nanocrystal compound of claim 10, wherein r is 2, 3, 4, or 5.

13. The nanocrystal compound of claim 10, wherein the compound is 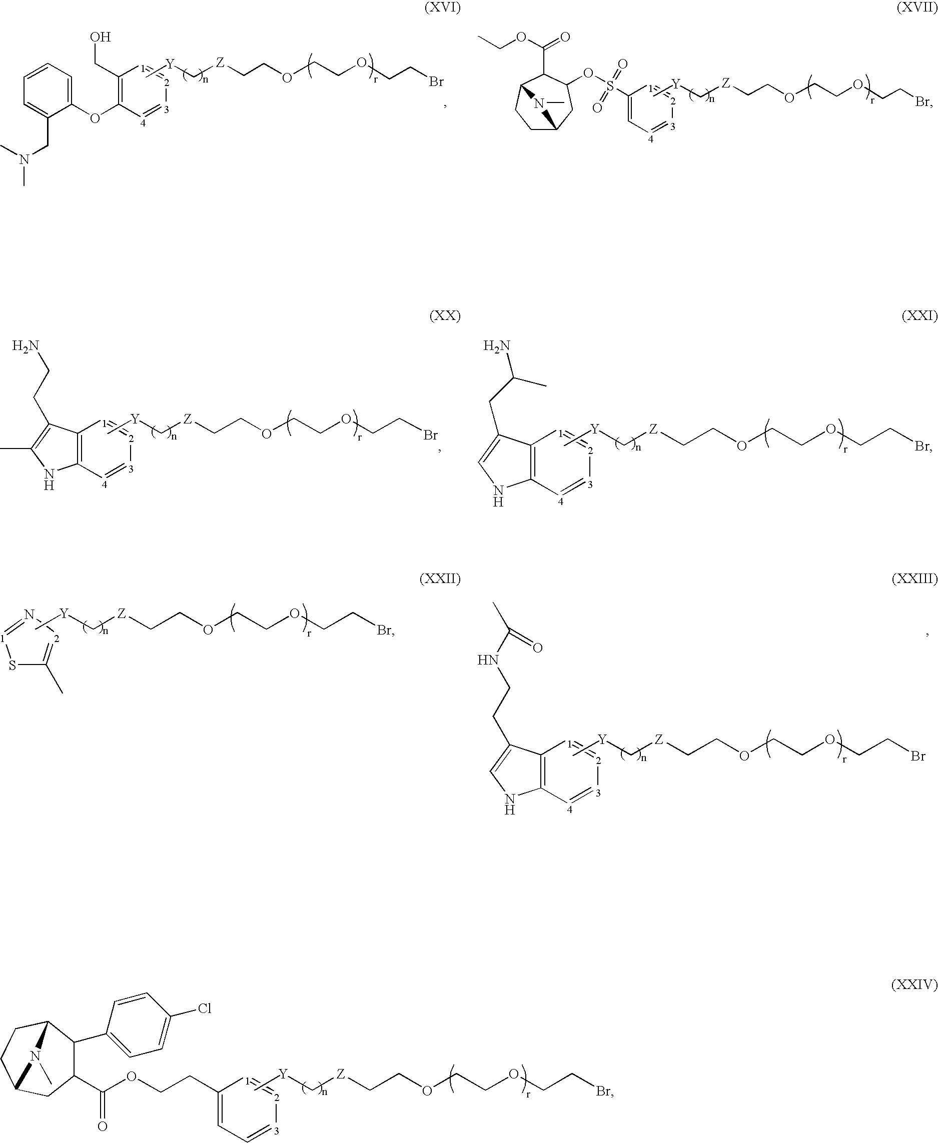 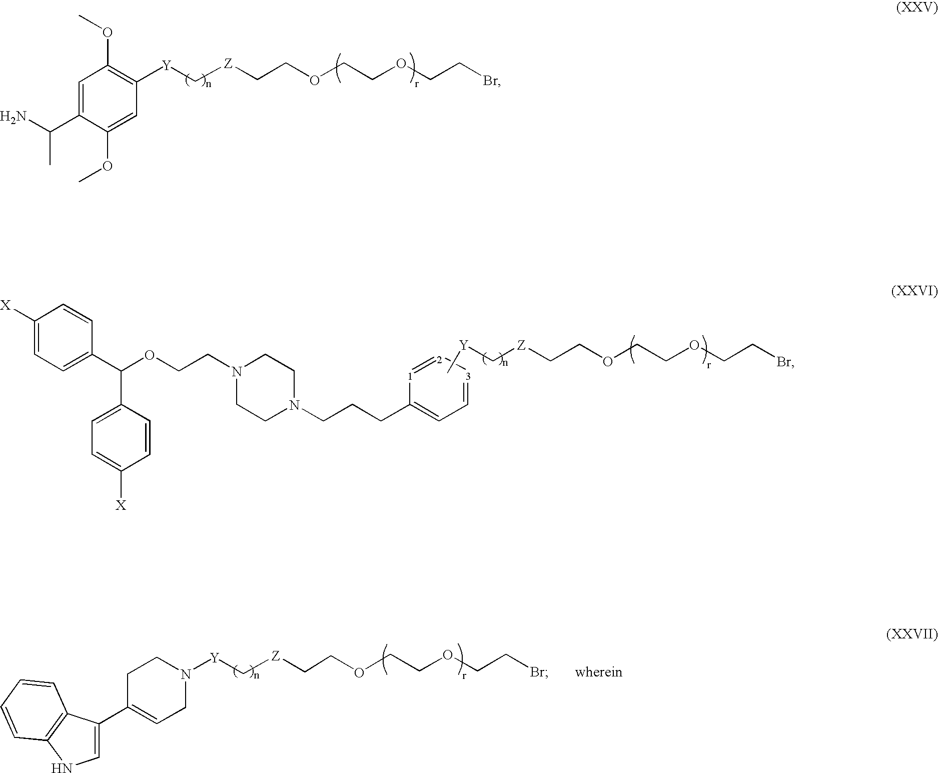
r is 1-10;
Y and Z are independently O, S, NH, CH₂, CONH, NHCO, NH, SO, SO₂NH, NHSO₂, carbamate, thiocarbamate, NH—R;
R is aryl or alkyl; and
n is 1-15.

14. The nanocrystal compound of claim 13, wherein r is 2, 3, 4, or 5.

15. The nanocrystal compound of claim 13, wherein n is 4, 5, 6, 7, 8, 9, or 10.
